(19) United States
(12) Patent Application Publication
(10) Pub. No.: US 2024/0124503 A1
(43) Pub. Date: Apr. 18, 2024

(54) HETEROAROMATIC PHOSPHONIUM SALTS AND THEIR USE TREATING CANCER

(71) Applicant: FLORATEK PHARMA S.A., Aubonne (CH)

(72) Inventor: Dan Florin Stoicescu, Aubonne (CH)

(21) Appl. No.: 18/274,314

(22) PCT Filed: Jan. 26, 2022
(86) PCT No.: PCT/EP2022/051808

§ 371 Date: Jul. 26, 2023

(30) Foreign Application Priority Data
Jan. 26, 2021 (GB) 2101043.4

(51) Int. Cl. C07F 9/655; A61P 35/00

(52) U.S. Cl. CPC C07F 9/65522; A61P 35/00

(57) ABSTRACT

The present invention relates to chromen-4-one derivatives comprising a phosphonium quaternary group, and to associated multi-salts, solvates, prodrugs and pharmaceutical compositions. The present invention also relates to the use of such compounds and compositions in the treatment and prevention of cancer.

### FIELD OF THE INVENTION

The present invention relates to flavonoid compounds, and to associated multi-salts, solvates, prodrugs and pharmaceutical compositions. The present invention also relates to the use of such compounds and compositions in the treatment and prevention of cancer.

### BACKGROUND

Targeting delayed or inhibited apoptosis is a major approach in cancer treatment and a highly active area of research. Apoptosis is a stringently organized process, regulated by a series of signal transduction cascades and cellular proteins. Two major pathways contributing to apoptosis: firstly, the extrinsic/death receptor induced pathway and secondly, the intrinsic pathway in which mitochondrial stress is involved [Rathore R., McCallum J. E., Varghese E., Maria A., Büsselberg D. Overcoming chemotherapy drug resistance by targeting inhibitors of apoptosis proteins (iaps) Apoptosis. 2017; 22:898-919]. Mitochondrial pathway of apoptosis is the most commonly deregulated type of cell death in cancer, and the understanding of mitochondrial apoptosis had advanced, so that novel therapies can be developed to specifically activate this process. [Lopez J., Tait S. W. G. Mitochondrial apoptosis: Killing cancer using the enemy within. Br. J. Cancer. 2015; 112:957-962]. In healthy cells, mitochondria execute a controlled regulation of multiple functions to maintain the cellular growth-death cycle. However, in the case of tumour cells, to meet the higher metabolic demand of rapidly proliferating cells, dysregulation of mitochondrial metabolism occurs. The difference between cancer cell mitochondria and normal cells includes several functional alterations, such as mutation of mtDNA, deficient respiration and ATP generation, mutation of mtDNA-encoded mitochondrial enzymes and structural differences, such as higher membrane potential of cancer cell mitochondria and higher basicity inside the mitochondrial lumen. The evasion of cell death or inhibition of mitochondria-mediated apoptosis is a hallmark for cancer. Mitochondria generate ROS, which is necessary for signalling under normal conditions. However, when apoptosis is inhibited in the case of cancer, ROS contributes to the neoplastic transformation. This altered mitochondrial metabolism of cancer cells compared with that of their normal counterparts is advantageous for the selective targeting of cancer mitochondria in therapeutics, which focuses on the cancer mitochondria specific features [Rin Jean S., Tulumello D. V., Wisnovsky S. P., Lei E. K., Pereira M. P., Kelley S. O. Molecular vehicles for mitochondrial chemical biology and drug delivery. ACS Chem. Biol. 2014; 9:323-333]. Anticancer drugs that selectively disrupt cancerous mitochondria could be achieved by designing molecules that act on the malignant mitochondria by, for instance, inhibiting glycolysis, depolarizing the membrane potential, and inhibiting the mitochondrial permeability transition pore [Dilip A., Cheng G., Joseph J., Kunnimalaiyaan S., Kalyanaraman B., Kunnimalaiyaan M., Gamblin T. C. Mitochondria-targeted antioxidant and glycolysis inhibition: Synergistic therapy in hepatocellular carcinoma. Anticancer Drugs. 2013; 24:881-888].

There is a need to provide compounds with improved pharmacological and/or physiological and/or physiochemical properties and/or those that provide a useful alternative to known compounds.

### SUMMARY OF THE INVENTION

The present invention addresses the limitations of the polyphenol class of compounds in maximizing their natural anti-cancer potential by providing a series of structurally novel compounds targeted to the mitochondrial membrane, thus enhancing the apoptotic pathway and potentially overcoming drug resistance by bypassing the cells mechanism of evading the apoptotic pathway. The compounds are effective through a multi-targeted approach using the lipophilic ion to rapidly penetrate and accumulate in the mitochondrial membrane and the polyphenolic moiety to exert anti-oxidant and antiproliferative effects. Additionally or alternatively, the discovered compound series optimizes the alkyl linker used to connect the lipophilic ion with the biologically active moiety.

The present invention is defined in the claims.

A first aspect of the invention provides a compound of formula (I):

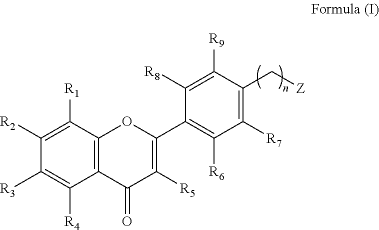

wherein:
Z is —[P(R¹¹)₃]X, wherein X is a counter anion;
R¹, R², and R⁵, independently, are selected from —OH, —O—C₁₋₄ alkyl, —OC(O)R₁₃, —OC(O)NHR¹³, and —OC(O)N(R¹³)₂; or
R¹ and R² together form —O—(C₁₋₃ alkylene)-O—; and R⁵ is selected from —OH, —O—C₁₋₄ alkyl, —OC(O)R₁₃, —OC(O)NHR¹³, and —OC(O)N(R¹³)₂;
R³, R⁴, R⁶, R⁷, R⁸, and R⁹, independently, are selected from H; halo; —CN; —NO₂; —R^{β}; —OH, —OR^{β}; —SH; —SR^{β}; —SOR^{β}; —SO₂H; —SO₂R^{β}; —SO₂NH₂; —SO₂NHR^{β}; —SO₂N(R^{β})₂; —NH₂; —NHR^{β}; —N(R^{β})₂; —CHO; —COR^{β}; —COOH; —COOR^{β}; —OCOR^{β}; and benzyl optionally substituted with 1-3—R^{β};
each —R^{β} is independently selected from a C₁-C₆ alkyl, C₂-C₆ alkenyl, C₂-C₆ alkynyl or C₃-C₁₄ cyclic group, and wherein any —R^{β} may optionally be substituted with one or more C₁-C₄ alkyl, C₁-C₄ haloalkyl, C₃-C₇ cycloalkyl, —O(C₁-C₄ alkyl), —O(C₁-C₄ haloalkyl), —O(C₃-C₇ cycloalkyl), halo, —OH, —NH₂, —CN, —NO₂, —C≡CH, —CHO, —CON(CH₃)₂ or oxo (═O) groups;
each —R¹¹ is independently selected from H, C₁-C₆ alkyl, C₂-C₆ alkenyl, C₃-C₁₄ aryl group, or C₃-C₁₄ aliphatic cyclic group, and wherein any —R¹¹ may optionally be substituted with one or more C₁-C₄ alkyl, C₁-C₄ haloalkyl, C₃-C₇ cycloalkyl, —O(C₁-C₄ alkyl), —O(C₁-C₄ haloalkyl), —O(C₃-C₇ cycloalkyl), halo, —OH, —NH₂, —CN, —C≡CH or oxo (═O) groups
each —R¹³ is independently selected from a H, C₁-C₆ alkyl, C₂-C₆ alkenyl, C₂-C₆ alkynyl, C₃₋₁₄ cyclic group, halo, —NO₂, —CN, —OH, —NH₂, mercapto, formyl, carboxy, carbamoyl, C₁₋₆ alkoxy, C₁₋₆ alkylthio, —NH(C₁₋₆ alkyl), —N(C₁₋₆ alkyl)₂, C₁₋₆ alkylsulfinyl, C₁₋₆ alkylsulfonyl, or arylsulfonyl, wherein any —R¹³ may optionally be substituted with one or more —R¹⁴;
each R¹⁴ is independently selected from a C₁-C₆ alkyl, C₂-C₆ alkenyl, C₂-C₆ alkynyl, C₃₋₁₄ cyclic group, halo, —NO₂, —CN, —OH, —NH₂, mercapto, formyl, carboxy, carbamoyl, C₁₋₆ alkoxy, C₁₋₆ alkylthio, —NH(C₁₋₆ alkyl), —N(C₁₋₆ alkyl)₂, C₁₋₆ alkylsulfinyl, C₁₋₆ alkylsulfonyl, or arylsulfonyl, wherein any —R₁₄ may optionally be substituted with one or more —R₁₅;
each —R¹⁵ is independently selected from halogen, nitro, cyano, hydroxy, trifluoromethoxy, trifluoromethyl, amino, formyl, carboxy, carbamoyl, mercapto, sulfamoyl, methyl, ethyl, methoxy, ethoxy, acetyl, acetoxy, methylamino, ethylamino, dimethylamino, diethylamino, N-methyl-N-ethylamino, acetylamino, N-methylcarbamoyl N-ethylcarbamoyl N,N-dimethylcarbamoyl, N,N-diethylcarbamoyl, N-methyl-N-ethylcarbamoyl, methylthio, ethylthio, methylsulfinyl, ethylsulfinyl, mesyl ethylsulfonyl, methoxycarbonyl, ethoxycarbonyl, N-methylsulfamoyl N-ethylsulfamoyl N,N-dimethylsulfamoyl N,N-diethylsulfamoyl, N-methyl-N-ethylsulfamoyl, carbocyclyl, aryl, or heterocyclyl;
n=1-10.

For example, n may be selected from an integer from 3 to 6.

For example, the compound may be a compound of Formula 1A:

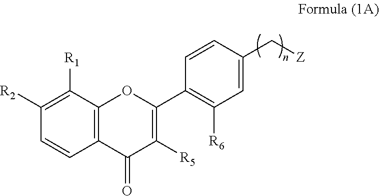

wherein:
Z is —[P(R¹¹)₃]X, wherein X is a counter anion;
R¹, R², and R⁵, independently, are selected from —OH, —O—C₁₋₄ alkyl, —OC(O)R₁₃, —OC(O)NHR¹³, —OC(O)N(R¹³)₂; or
R¹ and R² together form —O—(C₁₋₃ alkylene)-O—; and R⁵ is selected from —OH, —O—C₁₋₄ alkyl, —OC(O)R₁₃, —OC(O)NHR¹³, and —OC(O)N(R¹³)₂;
R⁶ is selected from H; halo; —CN; —NO₂; —R^{β}; —OH, —OR^{β}; —SH; —SR^{β}; —SOR^{β}; —SO₂H; —SO₂R^{β}; —SO₂NH₂; —SO₂NHR^{β}; —SO₂N(R^{β})₂; —NH₂; —NHR^{β}; —N(R^{β})₂; —CHO; —COR^{β}; —COOH; —COOR^{β}; —OCOR^{β}; and benzyl optionally substituted with 1-3—R^{β};
each —R^{β} is independently selected from a C₁-C₆ alkyl, C₂-C₆ alkenyl, C₂-C₆ alkynyl or C₃-C₁₄ cyclic group, and wherein any —R^{β} may optionally be substituted with one or more C₁-C₄ alkyl, C₁-C₄ haloalkyl, C₃-C₇ cycloalkyl, —O(C₁-C₄ alkyl), —O(C₁-C₄ haloalkyl), —O(C₃-C₇ cycloalkyl), halo, —OH, —NH₂, —CN, —NO₂, —C≡CH, —CHO, —CON(CH₃)₂ or oxo (═O) groups;
each —R¹¹ is independently selected from H, C₁-C₆ alkyl, C₂-C₆ alkenyl, C₃-C₁₄ aryl group, or C₃-C₁₄ aliphatic cyclic group, and wherein any —R¹¹ may optionally be substituted with one or more C₁-C₄ alkyl, C₁-C₄ haloalkyl, C₃-C₇ cycloalkyl, —O(C₁-C₄ alkyl), —O(C₁-C₄ haloalkyl), —O(C₃-C₇ cycloalkyl), halo, —OH, —NH₂, —CN, —C≡CH or oxo (═O) groups
each —R¹³ is independently selected from a H, C₁-C₆ alkyl, C₂-C₆ alkenyl, C₂-C₆ alkynyl, C₃₋₁₄ cyclic group, halo, —NO₂, —CN, —OH, —NH₂, mercapto, formyl, carboxy, carbamoyl, C₁₋₆ alkoxy, C₁₋₆ alkylthio, —NH(C₁₋₆ alkyl), —N(C₁₋₆ alkyl)₂, C₁₋₆ alkylsulfinyl, C₁₋₆ alkylsulfonyl, or arylsulfonyl, wherein any —R¹³ may optionally be substituted with one or more —R¹⁴;
each R¹⁴ is independently selected from a C₁-C₆ alkyl, C₂-C₆ alkenyl, C₂-C₆ alkynyl, C₃₋₁₄ cyclic group, halo, —NO₂, —CN, —OH, —NH₂, mercapto, formyl, carboxy, carbamoyl, C₁₋₆ alkoxy, C₁₋₆ alkylthio, —NH(C₁₋₆ alkyl), —N(C₁₋₆ alkyl)₂, C₁₋₆ alkylsulfinyl, C₁₋₆ alkylsulfonyl, or arylsulfonyl, wherein any —R₁₄ may optionally be substituted with one or more —R₁₅;
each —R¹⁵ is independently selected from halogen, nitro, cyano, hydroxy, trifluoromethoxy, trifluoromethyl, amino, formyl, carboxy, carbamoyl, mercapto, sulfamoyl, methyl, ethyl, methoxy, ethoxy, acetyl, acetoxy, methylamino, ethylamino, dimethylamino, diethylamino, N-methyl-N-ethylamino, acetylamino, N-methylcarbamoyl N-ethylcarbamoyl N,N-dimethylcarbamoyl, N,N-diethylcarbamoyl, N-methyl-N-ethylcarbamoyl, methylthio, ethylthio, methylsulfinyl, ethylsulfinyl, mesyl ethylsulfonyl, methoxycarbonyl, ethoxycarbonyl, N-methylsulfamoyl N-ethylsulfamoyl N,N-dimethylsulfamoyl N,N-diethylsulfamoyl, N-methyl-N-ethylsulfamoyl, carbocyclyl, aryl, or heterocyclyl;
n=1-10. For example, n may be selected from an integer between 3 and 6.

A second aspect of the invention provides a compound selected from the group consisting of:

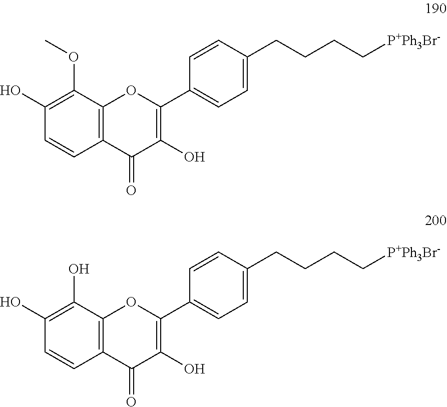

A third aspect of the invention provides pharmaceutically acceptable multi-salt, solvate or prodrug of the compound of the first or second aspect of the invention.

A fourth aspect of the invention provides a pharmaceutical composition comprising a compound of the first or second aspect of the invention, or a pharmaceutically acceptable multi-salt, solvate or prodrug of the third aspect of the invention, and a pharmaceutically acceptable excipient.

A fifth aspect of the invention provides a compound of the first or second aspect of the invention, or a pharmaceutically acceptable multi-salt, solvate or prodrug of the third aspect of the invention, or a pharmaceutical composition of the fourth aspect of the invention, for use in medicine, and/or for use in the treatment or prevention of a disease, disorder or condition. In one embodiment, the disease, disorder or condition is cancer.

A sixth aspect of the invention provides the use of a compound of the first or second aspect, a pharmaceutically effective multi-salt, solvate or prodrug of the third aspect, or a pharmaceutical composition according to the fourth aspect, in the manufacture of a medicament for the treatment or prevention of a disease, disorder or condition. Typically the treatment or prevention comprises the administration of the compound, multi-salt, solvate, prodrug or pharmaceutical composition to a subject. In one embodiment, the disease, disorder or condition is cancer.

A seventh aspect of the invention provides a method of treatment or prevention of a disease, disorder or condition, the method comprising the step of administering an effective amount of a compound of the first or second aspect, or a pharmaceutically acceptable multi-salt, solvate or prodrug of the third aspect, or a pharmaceutical composition of the fourth aspect, to thereby treat or prevent the disease, disorder or condition. Typically the administration is to a subject in need thereof. In one embodiment, the disease, disorder or condition is cancer.

### DETAILED DESCRIPTION OF THE INVENTION

A first aspect of the invention provides a compound of formula (I):

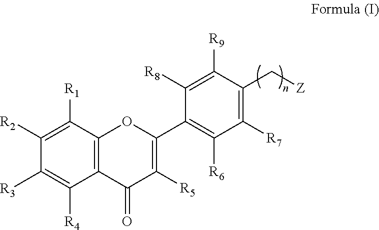

wherein:
Z is —[P(R¹¹)₃]X, wherein X is a counter anion;
R¹, R², and R⁵, independently, are selected from —OH, —O—C₁₋₄ alkyl, —OC(O)R₁₃, —OC(O)NHR¹³, —OC(O)N(R¹³)₂; or
R¹ and R² together form —O—(C₁₋₃ alkylene)-O—; and R⁵ is selected from —OH, —O—C₁₋₄ alkyl, —OC(O)R₁₃, —OC(O)NHR¹³, and —OC(O)N(R¹³)₂;
R³, R⁴, R⁶, R⁷, R⁸, and R⁹, independently, are selected from H; halo; —CN; —NO₂; —R^{β}; —OH, —OR^{β}; —SH; —SR^{β}; —SOR^{β}; —SO₂H; —SO₂R^{β}; —SO₂NH₂; —SO₂NHR^{β}; —SO₂N(R^{β})₂; —NH₂; —NHR^{β}; —N(R^{β})₂; —CHO; —COR^{β}; —COOH; —COOR^{β}; —OCOR^{β}; and benzyl optionally substituted with 1-3—R^{β};
each —R^{β} is independently selected from a C₁-C₆ alkyl, C₂-C₆ alkenyl, C₂-C₆ alkynyl or C₃-C₁₄ cyclic group, and wherein any —R^{β} may optionally be substituted with one or more C₁-C₄ alkyl, C₁-C₄ haloalkyl, C₃-C₇ cycloalkyl, —O(C₁-C₄ alkyl), —O(C₁-C₄ haloalkyl), —O(C₃-C₇ cycloalkyl), halo, —OH, —NH₂, —CN, —NO₂, —C≡CH, —CHO, —CON(CH₃)₂ or oxo (═O) groups;
each —R¹¹ is independently selected from H, C₁-C₆ alkyl, C₂-C₆ alkenyl, C₃-C₁₄ aryl group, or C₃-C₁₄ aliphatic cyclic group, and wherein any —R¹¹ may optionally be substituted with one or more C₁-C₄ alkyl, C₁-C₄ haloalkyl, C₃-C₇ cycloalkyl, —O(C₁-C₄ alkyl), —O(C₁-C₄ haloalkyl), —O(C₃-C₇ cycloalkyl), halo, —OH, —NH₂, —CN, —C≡CH or oxo (═O) groups
each —R¹³ is independently selected from a H, C₁-C₆ alkyl, C₂-C₆ alkenyl, C₂-C₆ alkynyl, C₃₋₁₄ cyclic group, halo, —NO₂, —CN, —OH, —NH₂, mercapto, formyl, carboxy, carbamoyl, C₁₋₆ alkoxy, C₁₋₆ alkylthio, —NH(C₁₋₆ alkyl), —N(C₁₋₆ alkyl)₂, C₁₋₆ alkylsulfinyl, C₁₋₆ alkylsulfonyl, or arylsulfonyl, wherein any —R¹³ may optionally be substituted with one or more —R¹⁴;
each R¹⁴ is independently selected from a C₁-C₆ alkyl, C₂-C₆ alkenyl, C₂-C₆ alkynyl, C₃₋₁₄ cyclic group, halo, —NO₂, —CN, —OH, —NH₂, mercapto, formyl, carboxy, carbamoyl, C₁₋₆ alkoxy, C₁₋₆ alkylthio, —NH(C₁₋₆ alkyl), —N(C₁₋₆ alkyl)₂, C₁₋₆ alkylsulfinyl, C₁₋₆ alkylsulfonyl, or arylsulfonyl, wherein any —R₁₄ may optionally be substituted with one or more —R₁₅;
each —R¹⁵ is independently selected from halogen, nitro, cyano, hydroxy, trifluoromethoxy, trifluoromethyl, amino, formyl, carboxy, carbamoyl, mercapto, sulfamoyl, methyl, ethyl, methoxy, ethoxy, acetyl, acetoxy, methylamino, ethylamino, dimethylamino, diethylamino, N-methyl-N-ethylamino, acetylamino, N-methylcarbamoyl N-ethylcarbamoyl N,N-dimethylcarbamoyl, N,N-diethylcarbamoyl, N-methyl-N-ethylcarbamoyl, methylthio, ethylthio, methylsulfinyl, ethylsulfinyl, mesyl ethylsulfonyl, methoxycarbonyl, ethoxycarbonyl, N-methylsulfamoyl N-ethylsulfamoyl N,N-dimethylsulfamoyl N,N-diethylsulfamoyl, N-methyl-N-ethylsulfamoyl, carbocyclyl, aryl, or heterocyclyl;
n=1-10.

In one embodiment, n=3-6.

In one embodiment, n is 3, 4, 5 or 6.

In one embodiment, n is 3 or 4.

In one embodiment, R¹, R², and R⁵, independently, are selected from —OH, —O—C₁₋₄ alkyl, —OC(O)R₁₃, —OC(O)NHR¹³, and —OC(O)N(R¹³)₂.

In one embodiment, R¹, R², and R⁵, independently, are selected from —OH, —O—C₁₋₄ alkyl, and —OC(O)R₁₃.

In one embodiment, R¹, R², and R⁵, independently, are selected from —OH, and —O—C₁₋₄ alkyl.

In one embodiment, R¹, R², and R⁵, independently, are selected from —OH, —OCH₃, —OC(O)C(CH₃)₃, —OC(O)NH—C₁₋₃ alkyl, and —OC(O)N(CH₃)₂, or R¹ and R² together form —O—CH₂—O—.

In one embodiment, R¹, R², and R⁵, independently, are selected from —OH, —OCH₃, —OC(O)C(CH₃)₃, —OC(O)NH—C₁₋₃ alkyl, and —OC(O)N(CH₃)₂.

In one embodiment, R¹, R², and R⁵, independently, are selected from —OH, and —O—C₁₋₄ alkyl. For example, R¹, R², and R⁵, independently, are selected from —OH, and —O—C₁₋₃ alkyl. For example, R¹, R², and R⁵, independently, are selected from —OH, and —O—C₁₋₂ alkyl. For example, R¹, R², and R⁵, independently, are selected from —OH and —O—CH₃.

In one embodiment, R¹, R², and R⁵, independently, are selected from —OH, —OCH₃, —OC(O)C(CH₃)₃, —OC(O)NH—C₁₋₃ alkyl, and —OC(O)N(CH₃)₂.

In one embodiment, R¹ and R², independently, are selected from —OH, —O—C₁₋₄ alkyl, —OC(O)R₁₃, —OC(O)NHR¹³, and —OC(O)N(R¹³)₂; and R⁵ is —OH.

In one embodiment, R¹ and R², independently, are selected from —OH, and —O—C₁₋₄ alkyl; and R⁵ is —OH.

In one embodiment, R¹ and R⁵, independently, are selected from —OH, —O—C₁₋₄ alkyl, —OC(O)R₁₃, —OC(O)NHR¹³, and —OC(O)N(R¹³)₂; and R² is —OH.

In one embodiment, R¹ and R⁵, independently, are selected from —OH, and —O—C₁₋₄ alkyl; and R² is —OH.

In one embodiment, Riis selected from —OH, —O—C₁₋₄ alkyl, —OC(O)R₁₃, —OC(O)NHR¹³, and —OC(O)N(R¹³)₂; and R² and R⁵ are —OH.

In one embodiment, Riis selected from —OH and —O—C₁₋₄ alkyl; and R² and R⁵ are —OH.

In one embodiment, R³, R⁴, R⁶, R⁷, R⁸, and R⁹, independently, are selected from H; halo; —CN; —NO₂; —R^{β}; —OH, —OR^{β}; —SH; —SR^{β}; —SOR^{β}; —SO₂H; —SO₂R^{β}; —SO₂NH₂; —SO₂NHR^{β}; —SO₂N(R^{β})₂; —NH₂; —NHR^{β}; —N(R^{β})₂; —CHO; —COR^{β}; —COOH; —COOR^{β}; —OCOR^{β}; and benzyl optionally substituted with 1-3—R^{β}.

In one embodiment, R³, R⁴, R⁶, R⁷, R⁸, and R⁹, independently, are selected from H; halo; —CN; —NO₂; —R^{β}; —SH; —SR^{β}; —SOR^{β}; —SO₂H; —SO₂R^{β}; —SO₂NH₂; —SO₂NHR^{β}; —SO₂N(R^{β})₂; —NH₂; —NHR^{β}; —N(R^{β})₂; —CHO; —COR^{β}; —COOH; and —COOR^{β}; and benzyl optionally substituted with 1-3—R^{β}.

In one embodiment, R³, R⁴, R⁶, R⁷, R⁸, and R⁹, independently, are selected from H; halo; —CN; —NO₂; —R^{β}; —NH₂; —NHR^{β}; —N(R^{β})₂; —CHO; —COR^{β}; —COOH; —COOR^{β}; and —OCOR^{β}.

In one embodiment, R³, R⁴, R⁶, R⁷, R⁸, and R⁹, independently, are selected from H; halo; —CN; —NO₂; —R^{β}; —NH₂; —NHR^{β}; —N(R^{β})₂; —CHO; —COR^{β}; —COOH; and —COOR^{β}.

In one embodiment, R³, R⁴, R⁶, R⁷, R⁸, and R⁹, independently, are selected from H; halo; —CN; —NO₂; —R^{β}; —NH₂; —NHR^{β}; —N(R^{β})₂; and —CHO.

In one embodiment, R³, R⁴, R⁶, R⁷, R⁸, and R⁹, independently, are selected from H; halo; —CN; —NO₂; —SH; —SO₂H; and —NH₂.

In one embodiment, R³, R⁴, R⁶, R⁷, R⁸, and R⁹ are H.

In one embodiment, R¹, R², and R⁵, independently, are selected from —OH, —O—C₁₋₄ alkyl, —OC(O)R₁₃, —OC(O)NHR¹³, and —OC(O)N(R¹³)₂; wherein R¹ and R² together may form —O—(C₁₋₃ alkylene)-O—; and R³, R⁴, R⁶, R⁷, R⁸, and R⁹, independently, are selected from H; halo; —CN; —NO₂; —R^{β}; —SH; —SR^{β}; —SOR^{β}; —SO₂H; —SO₂R^{β}; —SO₂NH₂; —SO₂NHR^{β}; —SO₂N(R^{β})₂; —NH₂; —NHR^{β}; —N(R^{β})₂; —CHO; —COR^{β}; —COOH; —COOR^{β}; —OCOR^{β}; and benzyl optionally substituted with 1-3—R^{β}.

In one embodiment, R¹, R², and R⁵, independently, are selected from —OH, —O—C₁₋₄ alkyl, —OC(O)R₁₃, —OC(O)NHR¹³, and —OC(O)N(R¹³)₂; and R³, R⁴, R⁶, R⁷, R⁸, and R⁹, independently, are selected from H; halo; —CN; —NO₂; —R^{β}; —SH; —SR^{β}; —SOR^{β}; —SO₂H; —SO₂R^{β}; —SO₂NH₂; —SO₂NHR^{β}; —SO₂N(R^{β})₂; —NH₂; —NHR^{β}; —N(R^{β})₂; —CHO; —COR^{β}; —COOH; —COOR^{β}; —OCOR^{β}; and benzyl optionally substituted with 1-3—R^{β}.

In one embodiment, R¹, R², and R⁵, independently, are selected from —OH, —O—C₁₋₄ alkyl, —OC(O)R₁₃, —OC(O)NHR¹³, —OC(O)N(R¹³)₂; and R³, R⁴, R⁶, R⁷, R⁸, and R⁹, independently, are selected from H; halo; —CN; —NO₂; —R^{β}; —OH, —OR^{β}; —SH; —SR^{β}; —SOR^{β}; —SO₂H; —SO₂R^{β}; —SO₂NH₂; —SO₂NHR^{β}; —SO₂N(R^{β})₂; —NH₂; —NHR^{β}; —N(R^{β})₂; —CHO; —COR^{β}; —COOH; —COOR^{β}; —OCOR^{β}; and benzyl optionally substituted with 1-3—R^{β}. For example, R¹, R², and R⁵, independently, are selected from —OH, —OCH₃, —OC(O)C(CH₃)₃, —OC(O)NH—C₁₋₃ alkyl, and —OC(O)N(CH₃)₂; and R³, R⁴, R⁶, R⁷, R⁸, and R⁹, independently, are selected from H; halo; —CN; —NO₂; —SH; —SO₂H; and —NH₂. For example, R¹, R², and R⁵, independently, are selected from —OH, —OCH₃, —OC(O)C(CH₃)₃, —OC(O)NH—C₁₋₃ alkyl, and —OC(O)N(CH₃)₂; and R³, R⁴, R⁶, R⁷, R⁸, and R⁹ are H.

In one embodiment, R¹, R², and R⁵, independently, are selected from —OH, —O—C₁₋₄ alkyl, —OC(O)R₁₃, —OC(O)NHR¹³, —OC(O)N(R¹³)₂; and R³, R⁴, R⁶, R⁷, R⁸, and R⁹, independently, are selected from H; halo; —CN; —NO₂; —R^{β}; —OH, —OR^{β}; —SH; —SR^{β}; —SOR^{β}; —SO₂H; —SO₂R^{β}; —SO₂NH₂; —SO₂NHR^{β}; —SO₂N(R^{β})₂; —NH₂; —NHR^{β}; —N(R^{β})₂; —CHO; —COR^{β}; —COOH; —COOR^{β}; —OCOR^{β}; and benzyl optionally substituted with 1-3—R^{β}. For example, R¹, R², and R⁵, independently, are selected from —OH, and —OCH₃; and R³, R⁴, R⁶, R⁷, R⁸, and R⁹, independently, are selected from H; halo; —CN; —NO₂; —SH; —SO₂H; and —NH₂. For example, R¹, R², and R⁵, independently, are selected from —OH, and —OCH₃; and R³, R⁴, R⁶, R⁷, R⁸, and R⁹ are H.

In one embodiment, R¹ and R² together form a —O—(C₁₋₃ alkylene)-O-group. For example, R¹ and R² together form —O-(methylene)-O—.

In one embodiment, each —R^{β} is independently selected from a C₁-C₆ alkyl, C₂-C₆ alkenyl, C₂-C₆ alkynyl or C₃-C₁₄ cyclic group, and wherein any —R^{β} may optionally be substituted with one or more C₁-C₄ alkyl, C₁-C₄ haloalkyl, C₃-C₇ cycloalkyl, —O(C₁-C₄ alkyl), —O(C₁-C₄ haloalkyl), —O(C₃-C₇ cycloalkyl), halo, —OH, —NH₂, —CN, —NO₂, —C≡CH, —CHO, —CON(CH₃)₂ or oxo (═O) groups.

In one embodiment, RR is independently selected from a C₁-C₆ alkyl, C₂-C₆ alkenyl, C₂-C₆ alkynyl or C₃-C₁₄ cyclic group, and wherein any —R^{β} may optionally be substituted with one or more halo, —OH, —NH₂, —CN, —NO₂, —C≡CH, —CHO, —CON(CH₃)₂ or oxo (═O) groups.

In one embodiment, each —R^{β} is independently selected from a C₁-C₆ alkyl, C₂-C₆ alkenyl, C₂-C₆ alkynyl or C₃-C₁₄ cyclic group.

In one embodiment, each —R³ is independently selected from —CF₃ and —CHF₂.

In one embodiment, each R^{β} is independently selected from a methyl, ethyl, n-propyl, i-propyl, n-butyl, i-butyl, t-butyl, n-pentyl, ethenyl, propenyl, 1-butenyl, 2-butenyl, 1-pentenyl, 1-hexenyl, 1,3-butadienyl, 1,3-pentadienyl, 1,4-pentadienyl, 1,4-hexadienyl, ethynyl, propargyl, but-1-ynyl or but-2-ynyl group.

In one embodiment, each —R^{β} is independently selected from a methyl, ethyl, n-propyl, i-propyl, n-butyl, i-butyl, t-butyl, or n-pentyl group.

In one embodiment, X is a pharmaceutically acceptable counter anion. In one embodiment, X is selected from but not limited to halides (for example fluoride, chloride, bromide or iodide) or other inorganic anions (for example nitrate, perchlorate, sulfate, bisulfate, or phosphate) or organic anions (for example propanoate, butyrate, glycolate, lactate, mandelate, citrate, acetate, benzoate, salicylate, succinate, malate, tartrate, fumarate, maleate, hydroxymaleate, galactarate, gluconate, pantothenate, pamoate, methanesulfonate, trifluoromethanesulfonate, ethanesulfonate, 2-hydroxyethanesulfonate, benzenesulfonate, toluene-p-sulfonate, naphthalene-2-sulfonate, camphorsulfonate, ornithinate, glutamate or aspartate).

In one embodiment, X may be a fluoride, chloride, bromide or iodide.

In one embodiment, X is bromide or chloride.

In one embodiment, X is bromide.

In one embodiment, Z is —[P(R¹¹)₃]X, wherein each —R¹¹ is independently selected from H, C₁-C₆ alkyl, C₂-C₆ alkenyl, C₃-C₁₄ aryl group, or C₃-C₁₄ aliphatic cyclic group, and wherein any —R¹¹ may optionally be substituted with one or more C₁-C₄ alkyl, C₁-C₄ haloalkyl, C₃-C₇ cycloalkyl, —O(C₁-C₄ alkyl), —O(C₁-C₄ haloalkyl), —O(C₃-C₇ cycloalkyl), halo, —OH, —NH₂, —CN, —C≡CH or oxo (═O) groups; and wherein X is a counter anion. For example, X may be bromide or chloride.

In one embodiment, Z is —[P(R¹¹)₃]X, wherein each —R¹¹ is independently selected from H, C₁-C₆ alkyl, C₂-C₆ alkenyl, C₃-C₁₄ aryl group, or C₃-C₁₄ aliphatic cyclic group; and wherein X is a counter anion. For example, X may be bromide or chloride.

In one embodiment, Z is —[P(R¹¹)₃]X, wherein each —R¹¹ is independently selected from H, C₁-C₆ alkyl, C₂-C₆ alkenyl, C₃-C₁₄ aryl group, or C₃-C₁₄ aliphatic cyclic group, and wherein any —R¹¹ may optionally be substituted with one or more C₁-C₄ alkyl, C₁-C₄ haloalkyl, C₃-C₇ cycloalkyl, —O(C₁-C₄ alkyl), —O(C₁-C₄ haloalkyl), —O(C₃-C₇ cycloalkyl), halo, —OH, —NH₂, —CN, —C≡CH or oxo (═O) groups; and wherein X is a counter anion. For example, X may be bromide or chloride.

In one embodiment, Z is —[P(R¹¹)₃]X, wherein each —R¹¹ is independently selected from H, C₁-C₆ alkyl, C₂-C₆ alkenyl, C₃-C₁₄ aryl group, or C₃-C₁₄ aliphatic cyclic group; and wherein X is a counter anion. For example, X may be bromide or chloride.

In one embodiment, Z is —[P(R¹¹)₃]X, wherein each —R¹¹ is independently selected from H, C₁-C₆ alkyl, C₂-C₆ alkenyl, C₃-C₁₄ aryl group, or C₃-C₁₄ aliphatic cyclic group; and wherein X is a counter anion. For example, X may be bromide or chloride.

In one embodiment, Z is —[P(R¹)₃]X, wherein each —R¹¹ is independently selected from H, or C₁-C₆ alkyl, or C₃-C₁₄ aryl group; and wherein X is a counter anion. For example, X may be bromide or chloride.

In one embodiment, Z is —[P(R¹¹)₃]X, wherein each —R¹¹ is independently a C₃-C₁₄ aryl group; and wherein any —R¹¹ may optionally be substituted with one or more C₁-C₄ alkyl, halo, —OH, —NH₂, —CN, —C≡CH or oxo (═O) groups; and wherein X is a counter anion. For example, X may be bromide or chloride.

In one embodiment, two of the R¹¹ groups are the same. In one embodiment, each R¹¹ group is the same.

In one embodiment, each R¹¹ group is the same; preferably each R¹¹ is a phenyl group.

In one embodiment, Z is —[P(R¹¹)₃]X, wherein each —R¹¹ is a phenyl group; each phenyl group may optionally be substituted with one or more C₁-C₄ alkyl, halo, —OH, —NH₂, —CN, —C≡CH or oxo (═O) groups; and wherein X is a counter anion. For example, X may be bromide or chloride.

In one embodiment, each R¹¹ is a phenyl group.

In one embodiment, Z is —[P(Ph)₃]X, wherein X is a counter anion. For example, X may be bromide or chloride, or X may be bromide.

In one embodiment, each —R¹³ is independently selected from a C₁-C₆ alkyl, C₂-C₆ alkenyl, C₂-C₆ alkynyl, C₃₋₁₄ cyclic group, halo, —NO₂, —CN, —OH, —NH₂, mercapto, formyl, carboxy, carbamoyl, C₁₋₆ alkoxy, C₁₋₆ alkylthio, —NH(C₁₋₆ alkyl), —N(C₁₋₆ alkyl)₂, C₁₋₆ alkylsulfinyl, C₁₋₆ alkylsulfonyl, or arylsulfonyl, wherein any —R¹³ may optionally be substituted with one or more —R¹⁴.

In one embodiment, each —R¹³ is independently selected from a C₁-C₆ alkyl, C₂-C₆ alkenyl, C₂-C₆ alkynyl, C₃₋₁₄ cyclic group, halo, —NO₂, —CN, —OH, —NH₂, mercapto, formyl, carboxy, carbamoyl, C₁₋₆ alkoxy, C₁₋₆ alkylthio, —NH(C₁₋₆ alkyl), —N(C₁₋₆ alkyl)₂, C₁₋₆ alkylsulfinyl, C₁₋₆ alkylsulfonyl, or arylsulfonyl.

In one embodiment, each —R¹³ is independently selected from C₁₋₄ alkyl. For example, R¹³ is independently selected from C₁₋₃ alkyl.

In one embodiment, each —R¹³ is independently selected from a H, methyl, ethyl, n-propyl, i-propyl, n-butyl, i-butyl, t-butyl, n-pentyl, ethenyl, propenyl, 1-butenyl, 2-butenyl, 1-pentenyl, 1-hexenyl, 1,3-butadienyl, 1,3-pentadienyl, 1,4-pentadienyl, 1,4-hexadienyl, ethynyl, propargyl, but-1-ynyl or but-2-ynyl group.

In one embodiment, each —R¹³ is independently selected from H, methyl, ethyl, n-propyl, i-propyl, n-butyl, i-butyl, t-butyl, or n-pentyl group.

In one embodiment, each —R¹³ is independently selected from H, methyl, ethyl, propyl, and butyl.

In one embodiment, each R¹⁴ is independently selected from a C₁-C₆ alkyl, C₂-C₆ alkenyl, C₂-C₆ alkynyl, C₃₋₁₄ cyclic group, halo, —NO₂, —CN, —OH, —NH₂, mercapto, formyl, carboxy, carbamoyl, C₁₋₆ alkoxy, C₁₋₆ alkylthio, —NH(C₁₋₆ alkyl), —N(C₁₋₆ alkyl)₂, C₁₋₆ alkylsulfinyl, C₁₋₆ alkylsulfonyl, or arylsulfonyl, wherein any —R₁₄ may optionally be substituted with one or more —R₁₅.

In one embodiment, each R¹⁴ is independently selected from a halo, —NO₂, —CN, —OH, —NH₂, mercapto, formyl, carboxy, or carbamoyl group.

In one embodiment, each —R¹⁴ is independently selected from methyl, ethyl, n-propyl, i-propyl, n-butyl, i-butyl, t-butyl, n-pentyl, ethenyl, propenyl, 1-butenyl, 2-butenyl, 1-pentenyl, 1-hexenyl, 1,3-butadienyl, 1,3-pentadienyl, 1,4-pentadienyl, 1,4-hexadienyl, ethynyl, propargyl, but-1-ynyl or but-2-ynyl.

In one embodiment, each —R¹⁴ is independently selected from a methyl, ethyl, n-propyl, i-propyl, n-butyl, i-butyl, t-butyl, or n-pentyl group.

In one embodiment, each —R¹⁵ is independently selected from halogen, nitro, cyano, hydroxy, trifluoromethoxy, trifluoromethyl, amino, formyl, carboxy, carbamoyl, mercapto, sulfamoyl, methyl, ethyl, methoxy, ethoxy, acetyl, acetoxy, methylamino, ethylamino, dimethylamino, diethylamino, N-methyl-N-ethylamino, acetylamino, N-methylcarbamoyl N-ethylcarbamoyl N,N-dimethylcarbamoyl, N,N-diethylcarbamoyl, N-methyl-N-ethylcarbamoyl, methylthio, ethylthio, methylsulfinyl, ethylsulfinyl, mesyl ethylsulfonyl, methoxycarbonyl, ethoxycarbonyl, N-methylsulfamoyl N-ethylsulfamoyl N,N-dimethylsulfamoyl N,N-diethylsulfamoyl, N-methyl-N-ethylsulfamoyl, carbocyclyl, aryl, or heterocyclyl.

In one embodiment, n is an integer from 3 to 5. In one embodiment, n is an integer from 4 to 6. In one embodiment, n is 3, 4, 5, or 6. In one embodiment, n is 3. In one embodiment, n is 4.

In one embodiment, R¹, R², and R⁵, are independently selected from —OH, —OCH₃, —OCO^{t}Bu, —OCONHCH₃, —OCONHCH₂CH₃ and —OCON(CH₃)₂, wherein R¹ and R² together may form —O—CH₂—O—; R³, R⁴, R⁶, R⁷, R⁸, and R⁹ are each H; Z is —[P(R¹¹)₃]X, wherein each —R¹¹ is a phenyl group; each phenyl group may optionally be substituted with one or more C₁-C₄ alkyl, halo, —OH, —NH₂, —CN, —C≡CH or oxo (═O) groups; X is a counter anion; and n is 3 or 4. For example, X may be bromide or chloride, or X may be bromide.

In one embodiment, R¹, R², and R⁵, are independently selected from —OH, —OCH₃, —OCO^{t}Bu, —OCONHCH₃, —OCONHCH₂CH₃ or —OCON(CH₃)₂, wherein R¹ and R² together may form —O—CH₂—O—; R³, R⁴, R⁶, R⁷, R⁸, and R⁹ are each H; Z is —[P(Ph)₃]X; X is a counter anion; and n is 3 or 4. For example, X may be bromide or chloride, or X may be bromide.

In one embodiment, the compounds include a quaternary phosphonium group and X is a counter anion. Preferably, the counter anion X may be any pharmaceutically acceptable, non-toxic counter ion. For example, X may be bromide or chloride, or X may be bromide.

The counter anion may optionally be singly, doubly or triply charged. As the quaternary group is singly charged, if the counter anion is triply charged then the stoichiometric ratio of the quaternary group to counter anion will typically be 3:1 and if the counter anion is doubly charged then the stoichiometric ratio of the quaternary group to counter anion will typically be 2:1. If both the quaternary group and the counter anion are singly charged then the stoichiometric ratio of the quaternary group to counter anion will typically be 1:1.

In one embodiment, the counter anion will be a singly charged anion. Suitable anions X include but are not limited to halides (for example fluoride, chloride, bromide or iodide) or other inorganic anions (for example nitrate, perchlorate, sulfate, bisulfate, or phosphate) or organic anions (for example propanoate, butyrate, glycolate, lactate, mandelate, citrate, acetate, benzoate, salicylate, succinate, malate, tartrate, fumarate, maleate, hydroxymaleate, galactarate, gluconate, pantothenate, pamoate, methanesulfonate, trifluoromethanesulfonate, ethanesulfonate, 2-hydroxyethanesulfonate, benzenesulfonate, toluene-p-sulfonate, naphthalene-2-sulfonate, camphorsulfonate, ornithinate, glutamate or aspartate). The counter anion may be fluoride, chloride, bromide or iodide. For example, X may be bromide or chloride, or X may be bromide.

In one embodiment, R³, R⁴, R⁷, R⁸, and R⁹ are H; and R⁶ is selected from —OH, —O—C₁₋₄ alkyl, —OC(O)R₁₃, —OC(O)NHR¹³, —OC(O)N(R¹³)₂. This corresponds to a compound of formula (1A):

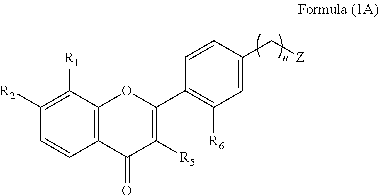

wherein R¹, R², R⁵, R⁶ and Z are as defined herein.

In one aspect of any of the above embodiments, the compound of formula (I) has a molecular weight of from 250 to 2,000 Da. Typically, the compound of formula (I) has a molecular weight of from 300 to 1,000 Da. Typically, the compound of formula (I) has a molecular weight of from 350 to 800 Da. More typically, the compound of formula (I) has a molecular weight of from 500 to 750 Da.

A second aspect of the invention provides a compound selected from the group consisting of:

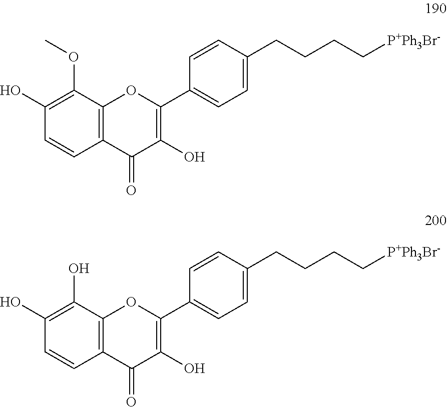

For example, the compound may be selected from the group consisting of:

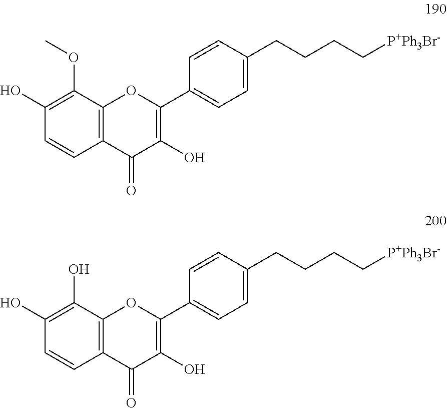

A third aspect of the invention provides a pharmaceutically acceptable multi-salt, solvate or prodrug of any compound of the first or second aspect of the invention.

The compounds of the present invention can be used both in their quaternary salt form (as a single salt). Additionally, the compounds of the present invention may contain one or more (e.g. one or two) acid addition or alkali addition salts to form a multi-salt. A multi-salt includes a quaternary salt group as well as a salt of a different group of the compound of the invention.

For the purposes of this invention, a “multi-salt” of a compound of the present invention includes an acid addition salt. Acid addition salts are preferably pharmaceutically acceptable, non-toxic addition salts with suitable acids, including but not limited to inorganic acids such as hydrohalogenic acids (for example, hydrofluoric, hydrochloric, hydrobromic or hydroiodic acid) or other inorganic acids (for example, nitric, perchloric, sulfuric or phosphoric acid); or organic acids such as organic carboxylic acids (for example, propionic, butyric, glycolic, lactic, mandelic, citric, acetic, benzoic, salicylic, succinic, malic or hydroxysuccinic, tartaric, fumaric, maleic, hydroxymaleic, mucic or galactaric, gluconic, pantothenic or pamoic acid), organic sulfonic acids (for example, methanesulfonic, trifluoromethanesulfonic, ethanesulfonic, 2-hydroxyethanesulfonic, benzenesulfonic, toluene-p-sulfonic, naphthalene-2-sulfonic or camphorsulfonic acid) or amino acids (for example, ornithinic, glutamic or aspartic acid). The acid addition salt may be a mono-, di-, tri- or multi-acid addition salt. A preferred salt is a hydrohalogenic, sulfuric, phosphoric or organic acid addition salt. A preferred salt is a hydrochloric acid addition salt.

The compounds of the present invention can be used both, in quaternary salt form and their multi-salt form. For the purposes of this invention, a “multi-salt” of a compound of the present invention includes one formed between a protic acid functionality (such as a carboxylic acid group) of a compound of the present invention and a suitable cation. Suitable cations include, but are not limited to lithium, sodium, potassium, magnesium, calcium and ammonium. The salt may be a mono-, di-, tri- or multi-salt. Preferably the salt is a mono- or di-lithium, sodium, potassium, magnesium, calcium or ammonium salt. More preferably the salt is a mono- or di-sodium salt or a mono- or di-potassium salt.

Preferably any multi-salt is a pharmaceutically acceptable non-toxic salt. However, in addition to pharmaceutically acceptable multi-salts, other salts are included in the present invention, since they have potential to serve as intermediates in the purification or preparation of other, for example, pharmaceutically acceptable salts, or are useful for identification, characterisation or purification of the free acid or base.

The compounds and/or multi-salts of the present invention may be anhydrous or in the form of a hydrate (e.g. a hemihydrate, monohydrate, dihydrate or trihydrate) or other solvate. Such solvates may be formed with common organic solvents, including but not limited to, alcoholic solvents e.g. methanol, ethanol or isopropanol.

In some embodiments of the present invention, therapeutically inactive prodrugs are provided. Prodrugs are compounds which, when administered to a subject such as a human, are converted in whole or in part to a compound of the invention. In most embodiments, the prodrugs are pharmacologically inert chemical derivatives that can be converted in vivo to the active drug molecules to exert a therapeutic effect. Any of the compounds described herein can be administered as a prodrug to increase the activity, bioavailability, or stability of the compound or to otherwise alter the properties of the compound. Typical examples of prodrugs include compounds that have biologically labile protecting groups on a functional moiety of the active compound.

Prodrugs include, but are not limited to, compounds that can be oxidized, reduced, aminated, deaminated, hydroxylated, dehydroxylated, hydrolyzed, dehydrolyzed, alkylated, dealkylated, acylated, deacylated, phosphorylated, and/or dephosphorylated to produce the active compound. The present invention also encompasses multi-salts and solvates of such prodrugs as described above.

The compounds, multi-salts, solvates and prodrugs of the present invention may contain at least one chiral centre. The compounds, multi-salts, solvates and prodrugs may therefore exist in at least two isomeric forms. The present invention encompasses racemic mixtures of the compounds, multi-salts, solvates and prodrugs of the present invention as well as enantiomerically enriched and substantially enantiomerically pure isomers. For the purposes of this invention, a “substantially enantiomerically pure” isomer of a compound comprises less than 5% of other isomers of the same compound, more typically less than 2%, and most typically less than 0.5% by weight.

The compounds, multi-salts, solvates and prodrugs of the present invention may contain any stable isotope including, but not limited to ¹²C, ¹³C, ¹H, ²H (D), ¹⁴N, ¹⁵N, ¹⁶O, ¹⁷O, ¹⁸O, ¹⁹F and ¹²⁷I, and any radioisotope including, but not limited to ¹¹C, ¹⁴C, ³H (T), ¹³N, ¹⁵O, ¹⁸F, ¹²³I, ¹²⁴I, ¹²⁵I and ¹³¹I.

The compounds, multi-salts, solvates and prodrugs of the present invention may be in any polymorphic or amorphous form.

A fourth aspect of the invention provides a pharmaceutical composition comprising a compound of the first or second aspect of the invention, or a pharmaceutically acceptable multi-salt, solvate or prodrug of the third aspect of the invention, and a pharmaceutically acceptable excipient.

Conventional procedures for the selection and preparation of suitable pharmaceutical formulations are described in, for example, “Aulton's Pharmaceutics—The Design and Manufacture of Medicines”, M. E. Aulton and K. M. G. Taylor, Churchill Livingstone Elsevier, 4^{th} Ed., 2013.

Pharmaceutically acceptable excipients including adjuvants, diluents or carriers that may be used in the pharmaceutical compositions of the invention are those conventionally employed in the field of pharmaceutical formulation, and include, but are not limited to, sugars, sugar alcohols, starches, ion exchangers, alumina, aluminium stearate, lecithin, serum proteins such as human serum albumin, buffer substances such as phosphates, glycerine, sorbic acid, potassium sorbate, partial glyceride mixtures of saturated vegetable fatty acids, water, salts or electrolytes such as protamine sulfate, disodium hydrogen phosphate, potassium hydrogen phosphate, sodium chloride, zinc salts, colloidal silica, magnesium trisilicate, polyvinylpyrrolidone, cellulose-based substances, polyethylene glycol, sodium carboxymethylcellulose, polyacrylates, waxes, polyethylene-polyoxypropylene-block polymers, polyethylene glycol and wool fat.

A fifth aspect of the invention provides a compound of the first or second aspect of the invention, or a pharmaceutically acceptable multi-salt, solvate or prodrug of the third aspect of the invention, or a pharmaceutical composition of the fourth aspect of the invention, for use in medicine, and/or for use in the treatment or prevention of a disease, disorder or condition. Typically the use comprises the administration of the compound, multi-salt, solvate, prodrug or pharmaceutical composition to a subject.

An sixth aspect of the invention provides the use of a compound of the first or second aspect, a pharmaceutically effective multi-salt, solvate or prodrug of the third aspect, or a pharmaceutical composition according to the fourth aspect in the manufacture of a medicament for the treatment or prevention of a disease, disorder or condition. Typically the treatment or prevention comprises the administration of the compound, multi-salt, solvate, prodrug or pharmaceutical composition to a subject.

A seventh aspect of the invention provides a method of treatment or prevention of a disease, disorder or condition, the method comprising the step of administering an effective amount of a compound of the first or second aspect, or a pharmaceutically acceptable multi-salt, solvate or prodrug of the third aspect, or a pharmaceutical composition of the fourth aspect, to thereby treat or prevent the disease, disorder or condition. Typically the administration is to a subject in need thereof.

The term “treatment” as used herein refers equally to curative therapy, and ameliorating or palliative therapy. The term includes obtaining beneficial or desired physiological results, which may or may not be established clinically. Beneficial or desired clinical results include, but are not limited to, the alleviation of symptoms, the prevention of symptoms, the diminishment of extent of disease, the stabilisation (i.e., not worsening) of a condition, the delay or slowing of progression/worsening of a condition/symptoms, the amelioration or palliation of the condition/symptoms, and remission (whether partial or total), whether detectable or undetectable. The term “palliation”, and variations thereof, as used herein, means that the extent and/or undesirable manifestations of a physiological condition or symptom are lessened and/or time course of the progression is slowed or lengthened, as compared to not administering a compound, multi-salt, solvate, prodrug or pharmaceutical composition of the present invention. The term “prevention” as used herein in relation to a disease, disorder or condition, relates to prophylactic or preventative therapy, as well as therapy to reduce the risk of developing the disease, disorder or condition. The term “prevention” includes both the avoidance of occurrence of the disease, disorder or condition, and the delay in onset of the disease, disorder or condition. Any statistically significant avoidance of occurrence, delay in onset or reduction in risk as measured by a controlled clinical trial may be deemed a prevention of the disease, disorder or condition. Subjects amenable to prevention include those at heightened risk of a disease, disorder or condition as identified by genetic or biochemical markers. Typically, the genetic or biochemical markers are appropriate to the disease, disorder or condition under consideration and may include for example, beta-amyloid 42, tau and phosphor-tau.

In general embodiments, the disease, disorder or condition is cancer.

In one embodiment, the cancer is brain cancer, breast cancer, colon cancer, leukaemia, lung cancer, lymphoma, ovarian cancer, pancreatic cancer, prostate cancer, renal cancer and skin cancer (melanoma).

In one embodiment the cancer is brain cancer.

In one embodiment the cancer is breast cancer.

In one embodiment the cancer is colon cancer.

In one embodiment the cancer is leukaemia.

In one embodiment the cancer is lung cancer.

In one embodiment the cancer is lymphoma.

In one embodiment the cancer is ovarian cancer.

In one embodiment the cancer is pancreatic cancer.

In one embodiment the cancer is prostate cancer.

In one embodiment the cancer is ovarian renal cancer.

In one embodiment the cancer is skin cancer (melanoma).

Unless stated otherwise, in any aspect of the invention, the subject may be any human or other animal. Typically, the subject is a mammal, more typically a human or a domesticated mammal such as a cow, pig, lamb, goat, horse, cat, dog, etc. Most typically, the subject is a human.

Any of the medicaments employed in the present invention can be administered by oral, parental (including intravenous, subcutaneous, intramuscular, intradermal, intratracheal, intraperitoneal, intraarticular, intracranial and epidural), airway (aerosol), rectal, vaginal or topical (including transdermal, buccal, mucosal and sublingual) administration.

Typically, the mode of administration selected is that most appropriate to the disorder or disease to be treated or prevented.

For oral administration, the compounds, multi-salts, solvates or prodrugs of the present invention will generally be provided in the form of tablets, capsules, hard or soft gelatine capsules, caplets, troches or lozenges, as a powder or granules, or as an aqueous solution, suspension or dispersion.

Tablets for oral use may include the active ingredient mixed with pharmaceutically acceptable excipients such as inert diluents, disintegrating agents, binding agents, lubricating agents, sweetening agents, flavouring agents, colouring agents and preservatives. Suitable inert diluents include sodium and calcium carbonate, sodium and calcium phosphate, and lactose. Corn starch and alginic acid are suitable disintegrating agents. Binding agents may include starch and gelatine. The lubricating agent, if present, may be magnesium stearate, stearic acid or tale. If desired, the tablets may be coated with a material, such as glyceryl monostearate or glyceryl distearate, to delay absorption in the gastrointestinal tract. Tablets may also be effervescent and/or dissolving tablets.

Capsules for oral use include hard gelatine capsules in which the active ingredient is mixed with a solid diluent, and soft gelatine capsules wherein the active ingredient is mixed with water or an oil such as peanut oil, liquid paraffin or olive oil.

Powders or granules for oral use may be provided in sachets or tubs. Aqueous solutions, suspensions or dispersions may be prepared by the addition of water to powders, granules or tablets.

Any form suitable for oral administration may optionally include sweetening agents such as sugar, flavouring agents, colouring agents and/or preservatives.

Formulations for rectal administration may be presented as a suppository with a suitable base comprising, for example, cocoa butter or a salicylate.

Formulations suitable for vaginal administration may be presented as pessaries, tampons, creams, gels, pastes, foams or spray formulations containing in addition to the active ingredient such carriers as are known in the art to be appropriate.

For parenteral use, the compounds, multi-salts, solvates or prodrugs of the present invention will generally be provided in a sterile aqueous solution or suspension, buffered to an appropriate pH and isotonicity. Suitable aqueous vehicles include Ringer's solution and isotonic sodium chloride or glucose. Aqueous suspensions according to the invention may include suspending agents such as cellulose derivatives, sodium alginate, polyvinylpyrrolidone and gum tragacanth, and a wetting agent such as lecithin. Suitable preservatives for aqueous suspensions include ethyl and n-propyl p-hydroxybenzoate. The compounds of the invention may also be presented as liposome formulations.

For transdermal and other topical administration, the compounds, multi-salts, solvates or prodrugs of the invention will generally be provided in the form of ointments, cataplasms (poultices), pastes, powders, dressings, creams, plasters or patches.

Suitable suspensions and solutions can be used in inhalers for airway (aerosol) administration.

The dose of the compounds, multi-salts, solvates or prodrugs of the present invention will, of course, vary with the disorder or disease to be treated or prevented. In general, a suitable dose will be in the range of 0.01 to 500 mg per kilogram body weight of the recipient per day. The desired dose may be presented at an appropriate interval such as once every other day, once a day, twice a day, three times a day or four times a day. The desired dose may be administered in unit dosage form, for example, containing 1 mg to 50 g of active ingredient per unit dosage form.

An eighth aspect of the invention provides a method of treatment or prevention of a disease, disorder or condition, the method comprising the step of administering an effective amount of a compound according to formula (1) as defined herein, or a pharmaceutically acceptable multi-salt, solvate or prodrug thereof, to thereby treat or prevent the disease, disorder or condition. Typically the administration is to a subject in need thereof. In one embodiment, the disease, disorder or condition is cancer.

### Definitions

In the context of the present specification, a “hydrocarbyl” substituent group or a hydrocarbyl moiety in a substituent group only includes carbon and hydrogen atoms but, unless stated otherwise, does not include any heteroatoms, such as N, O or S, in its carbon skeleton. A hydrocarbyl group/moiety may be saturated or unsaturated (including aromatic), and may be straight-chained or branched, or be or include cyclic groups wherein, unless stated otherwise, the cyclic group does not include any heteroatoms, such as N, O or S, in its carbon skeleton. Examples of hydrocarbyl groups include alkyl, alkenyl, alkynyl, cycloalkyl, cycloalkenyl and aryl groups/moieties and combinations of all of these groups/moieties. Typically a hydrocarbyl group is a C₁-C₁₂ hydrocarbyl group. More typically a hydrocarbyl group is a C₁-C₁₀ hydrocarbyl group. A “hydrocarbylene” group is similarly defined as a divalent hydrocarbyl group.

An “alkyl” substituent group or an alkyl moiety in a substituent group may be linear or branched. Examples of alkyl groups/moieties include methyl, ethyl, n-propyl, i-propyl, n-butyl, i-butyl, t-butyl and n-pentyl groups/moieties. Unless stated otherwise, the term “alkyl” does not include “cycloalkyl”. Typically an alkyl group is a C₁-C₁₂ alkyl group. More typically an alkyl group is a C₁-C₆ alkyl group. An “alkylene” group is similarly defined as a divalent alkyl group.

An “alkenyl” substituent group or an alkenyl moiety in a substituent group refers to an unsaturated alkyl group or moiety having one or more carbon-carbon double bonds. Examples of alkenyl groups/moieties include ethenyl, propenyl, 1-butenyl, 2-butenyl, 1-pentenyl, 1-hexenyl, 1,3-butadienyl, 1,3-pentadienyl, 1,4-pentadienyl and 1,4-hexadienyl groups/moieties. Unless stated otherwise, the term “alkenyl” does not include “cycloalkenyl”. Typically an alkenyl group is a C₂-C₁₂ alkenyl group. More typically an alkenyl group is a C₂-C₆ alkenyl group. An “alkenylene” group is similarly defined as a divalent alkenyl group.

An “alkynyl” substituent group or an alkynyl moiety in a substituent group refers to an unsaturated alkyl group or moiety having one or more carbon-carbon triple bonds. Examples of alkynyl groups/moieties include ethynyl, propargyl, but-1-ynyl and but-2-ynyl. Typically an alkynyl group is a C₂-C₁₂ alkynyl group. More typically an alkynyl group is a C₂-C₆ alkynyl group. An “alkynylene” group is similarly defined as a divalent alkynyl group.

A “haloalkyl” substituent group or haloalkyl group in a substituent group refers to an alkyl, alkenyl, or alkynyl substituent group or moiety including one or more carbon atoms and one or more halo atoms, e.g. Cl, Br, I, or F. Each halo atom replaces a hydrogen of the alkyl, alkenyl, or alkynyl substituent group or moiety. Examples include —CH₂F—CHF₂, —CHI₂, —CHBr₂, —CHCl₂, —CF₃, —CH₂CF₃ and CF₂CH₃.

An “alkoxy” substituent group or alkoxy group in a substituent group refers to an alkyl, alkenyl, or alkynyl substituent group or moiety including one or more carbon atoms and one or more oxygen atoms. Each oxygen atom replaces a carbon atom (for example the terminal or bonding carbon) of the alkyl, alkenyl, or alkynyl substituent group or moiety. Examples include —OCH₃, —OCH₂CH₃, —OCH₂CH₂CH₃, and —OCH(CH₃)(CH₃).

An “alkylthio” substituent group or alkylthio group in a substituent group refers to an alkyl, alkenyl, or alkynyl substituent group or moiety including one or more carbon atoms and one or more sulphur atoms. Each sulphur atom replaces a carbon atom (for example the terminal or bonding carbon) of the alkyl, alkenyl, or alkynyl substituent group or moiety. Examples include —SCH₃, —SCH₂CH₃, —SCH₂CH₂CH₃, and —SCH(CH₃)(CH₃).

An “alkylsulfinyl” substituent group or alkylsulfinyl group in a substituent group refers to an alkyl, alkenyl, or alkynyl substituent group or moiety including one or more carbon atoms and one or more sulfinyl groups (—S(═O)—). Each sulfinyl group replaces a carbon atom (for example the terminal or bonding carbon) of the alkyl, alkenyl, or alkynyl substituent group or moiety. Examples include —S(═O)CH₃, —S(═O)CH₂CH₃, —S(═O)CH₂CH₂CH₃, and —S(═O)CH(CH₃)(CH₃).

An “alkylsulfonyl” substituent group or alkylsulfonyl group in a substituent group refers to an alkyl, alkenyl, or alkynyl substituent group or moiety including one or more carbon atoms and one or more sulfonyl groups (—SO₂—). Each sulfonyl group replaces a carbon atom (for example the terminal or bonding carbon) of the alkyl, alkenyl, or alkynyl substituent group or moiety. Examples include —SO₂(CH₃), —SO₂(CH₂CH₃), —SO₂(CH₂CH₂CH₃), and —SO₂(CH(CH₃)(CH₃)).

An “arylsulfonyl” substituent group or arylsulfonyl group in a substituent group refers to an aryl substituent group or moiety including one or more carbon atoms and one or more sulfonyl groups (—SO₂—). Each sulfonyl group replaces a carbon atom (for example the terminal or bonding carbon) of the alkyl, alkenyl, or alkynyl substituent group or moiety. Examples include —SO₂(CH₃), —SO₂(CH₂CH₃), —SO₂(CH₂CH₂CH₃), and —SO₂(CH(CH₃)(CH₃)).

A “cyclic” substituent group or a cyclic moiety in a substituent group refers to any hydrocarbyl ring, wherein the hydrocarbyl ring may be saturated or unsaturated and may include one or more heteroatoms, e.g. N, O or S, in its carbon skeleton. Examples of cyclic groups include aliphatic cyclic, cycloalkyl, cycloalkenyl, heterocyclic, aryl and heteroaryl groups as discussed below. A cyclic group may be monocyclic, bicyclic (e.g. bridged, fused or spiro), or polycyclic. Typically, a cyclic group is a 3- to 12-membered cyclic group, which means it contains from 3 to 12 ring atoms. More typically, a cyclic group is a 3- to 7-membered monocyclic group, which means it contains from 3 to 7 ring atoms.

A “heterocyclic” substituent group or a heterocyclic moiety in a substituent group refers to a cyclic group or moiety including one or more carbon atoms and one or more heteroatoms, e.g. N, O or S, in the ring structure. Examples of heterocyclic groups include heteroaryl groups as discussed below and non-aromatic heterocyclic groups such as azetidinyl, azetinyl, tetrahydrofuranyl, pyrrolidinyl, tetrahydrothiophenyl, tetrahydropyranyl, piperidinyl, piperazinyl, morpholinyl and thiomorpholinyl groups.

An “aliphatic cyclic” substituent group or aliphatic cyclic moiety in a substituent group refers to a hydrocarbyl cyclic group or moiety that is not aromatic. The aliphatic cyclic group may be saturated or unsaturated and may include one or more heteroatoms, e.g. N, O or S, in its carbon skeleton. Examples include cyclopropyl, cyclohexyl and morpholinyl. Unless stated otherwise, an aliphatic cyclic substituent group or moiety may include monocyclic, bicyclic or polycyclic hydrocarbyl rings.

A “cycloalkyl” substituent group or a cycloalkyl moiety in a substituent group refers to a saturated hydrocarbyl ring containing, for example, from 3 to 7 carbon atoms, examples of which include cyclopropyl, cyclobutyl, cyclopentyl and cyclohexyl. Unless stated otherwise, a cycloalkyl substituent group or moiety may include monocyclic, bicyclic or polycyclic hydrocarbyl rings.

A “cycloalkenyl” substituent group or a cycloalkenyl moiety in a substituent group refers to a non-aromatic unsaturated hydrocarbyl ring having one or more carbon-carbon double bonds and containing, for example, from 3 to 7 carbon atoms, examples of which include cyclopent-1-en-1-yl, cyclohex-1-en-1-yl and cyclohex-1,3-dien-1-yl. Unless stated otherwise, a cycloalkenyl substituent group or moiety may include monocyclic, bicyclic or polycyclic hydrocarbyl rings.

An “aryl” substituent group or an aryl moiety in a substituent group refers to an aromatic hydrocarbyl ring. The term “aryl” includes monocyclic aromatic hydrocarbons and polycyclic fused ring aromatic hydrocarbons wherein all of the fused ring systems (excluding any ring systems which are part of or formed by optional substituents) are aromatic. Examples of aryl groups/moieties include phenyl, naphthyl, anthracenyl and phenanthrenyl. Unless stated otherwise, the term “aryl” does not include “heteroaryl”.

A “heteroaryl” substituent group or a heteroaryl moiety in a substituent group refers to an aromatic heterocyclic group or moiety. The term “heteroaryl” includes monocyclic aromatic heterocycles and polycyclic fused ring aromatic heterocycles wherein all of the fused ring systems (excluding any ring systems which are part of or formed by optional substituents) are aromatic. Examples of heteroaryl groups/moieties include the following:

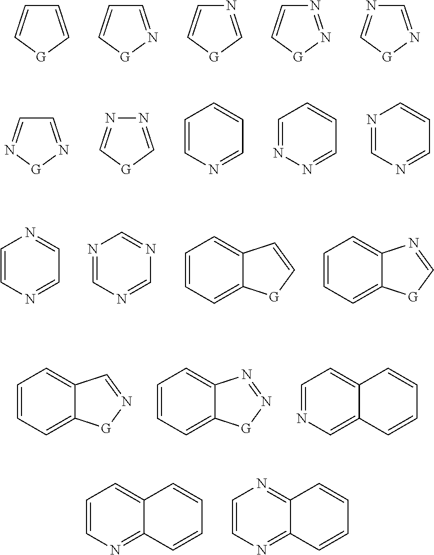

wherein G═O, S or NH.

For the purposes of the present specification, where a combination of moieties is referred to as one group, for example, arylalkyl, arylalkenyl, arylalkynyl, alkylaryl, alkenylaryl or alkynylaryl, the last mentioned moiety contains the atom by which the group is attached to the rest of the molecule. An example of an arylalkyl group is benzyl.

Typically a substituted group comprises 1, 2, 3 or 4 substituents, more typically 1, 2 or 3 substituents, more typically 1 or 2 substituents, and even more typically 1 substituent.

Unless stated otherwise, any divalent bridging substituent (e.g. —O—, —S—, —NH—, —N(R^{β})— or —R^{α}-) of an optionally substituted group or moiety must only be attached to the specified group or moiety and may not be attached to a second group or moiety, even if the second group or moiety can itself be optionally substituted.

The term “halo” includes fluoro, chloro, bromo and iodo.

Where reference is made to a carbon atom of a group being replaced by an N, O or S atom, what is intended is that:

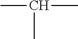

is replaced by

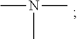

—CH₂— is replaced by —NH—, —O— or —S—;
—CH₃ is replaced by —NH₂, —OH, or —SH;
—CH═ is replaced by —N═;
CH₂═ is replaced by NH═, O═ or S═; or
CH≡ is replaced by N≡.

In the context of the present specification, unless otherwise stated, a Cₓ-C_{y} group is defined as a group containing from x to y carbon atoms. For example, a C₁-C₄ alkyl group is defined as an alkyl group containing from 1 to 4 carbon atoms. Optional substituents and moieties are not taken into account when calculating the total number of carbon atoms in the parent group substituted with the optional substituents and/or containing the optional moieties. For the avoidance of doubt, replacement heteroatoms, e.g. N, O or S, are counted as carbon atoms when calculating the number of carbon atoms in a Cₓ-C_{y} group. For example, a morpholinyl group is to be considered a C₆ heterocyclic group, not a C₄ heterocyclic group.

A “protecting group” refers to a grouping of atoms that when attached to a reactive functional group (e.g. OH) in a compound masks, reduces or prevents reactivity of the functional group.

In the context of the present specification, ═ is a double bond; ≡ is a triple bond.

The protection and deprotection of functional groups is described in ‘Protective Groups in Organic Synthesis’, 2^{nd} edition, T. W. Greene and P. G. M Wuts, Wiley-Interscience.

For the avoidance of doubt, insofar as is practicable any embodiment of a given aspect of the present invention may occur in combination with any other embodiment of the same aspect of the present invention. In addition, insofar as is practicable it is to be understood that any preferred, typical or optional embodiment of any aspect of the present invention should also be considered as a preferred, typical or optional embodiment of any other aspect of the present invention.

### EXAMPLES

The following nomenclature is used to refer to the following compounds.

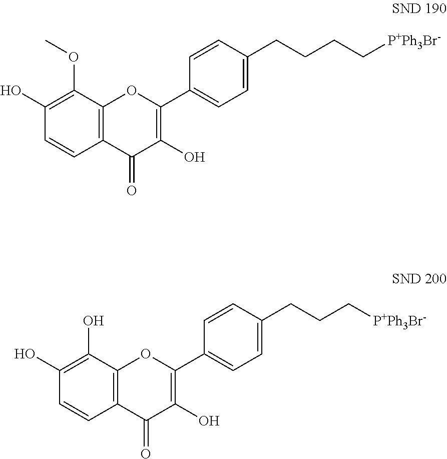

### Examples—Compound Synthesis

Compounds of the invention are synthesised employing a route of synthesis shown below. The general route of synthesis is illustrated below by reference to the synthesis of a specific compound. However, this is merely illustrative of a more general synthesis that can be employed to synthesise all compounds of the invention.

Route of Synthesis:

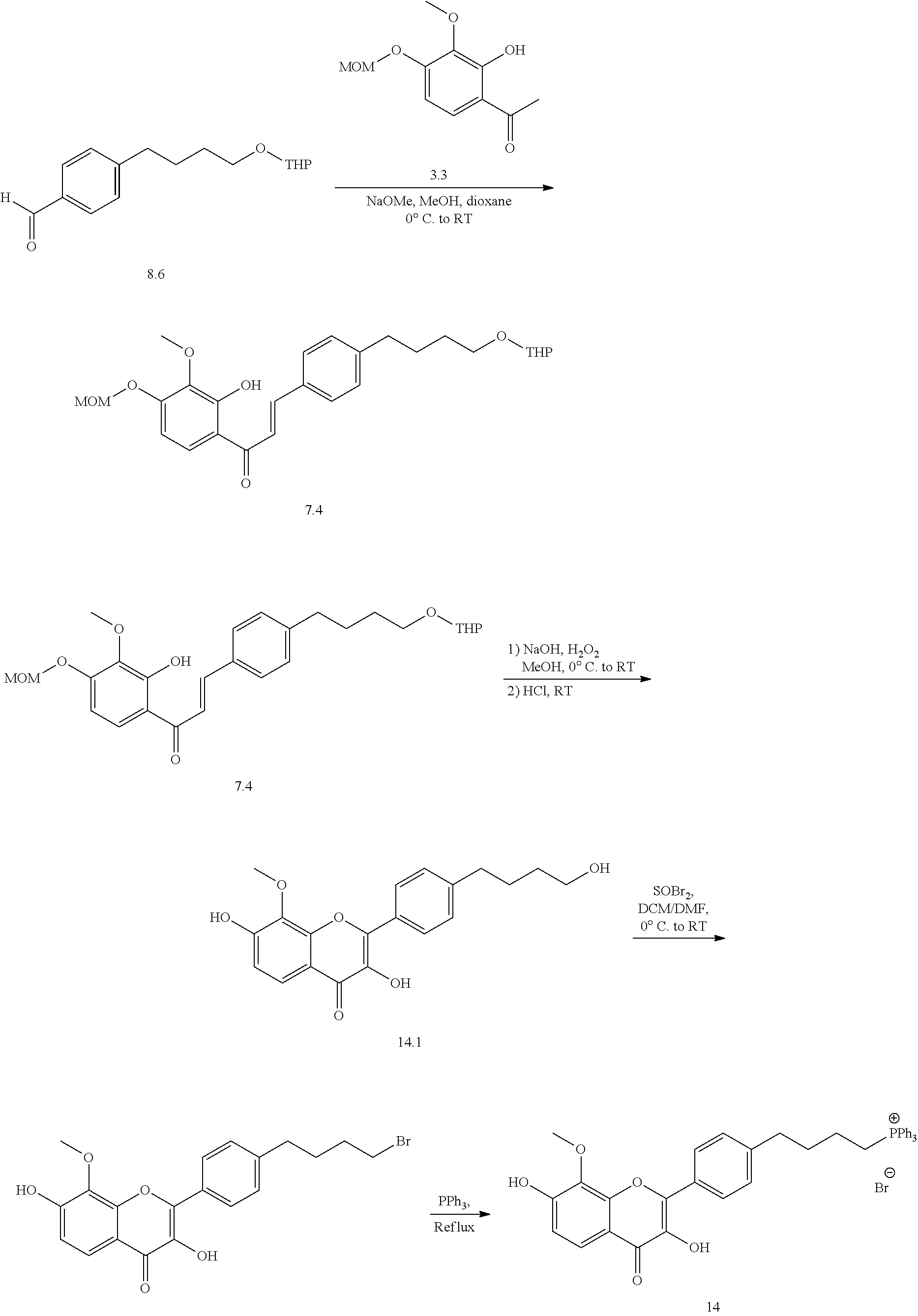

All solvents, reagents and compounds were purchased and used without further purification unless stated otherwise.

Abbreviations
LiHMDS—Lithium bis(trimethylsilyl)amide
THF—Tetrahydrofuran
THP—Tetrahydropyran
Pd/C—Palladium on carbon (10 wt. % loading)
AcOH—Acetic acid
DCM—Dichloromethane
MeOH—Methanol
EtOH—Ethanol
Et₂NH—Diethylamine
TsOH—Toluenesulfonic acid

### Synthesis Example 1: SND 190

The Following Route of Synthesis was Adopted to Prepare SND 190 (Compound 14):

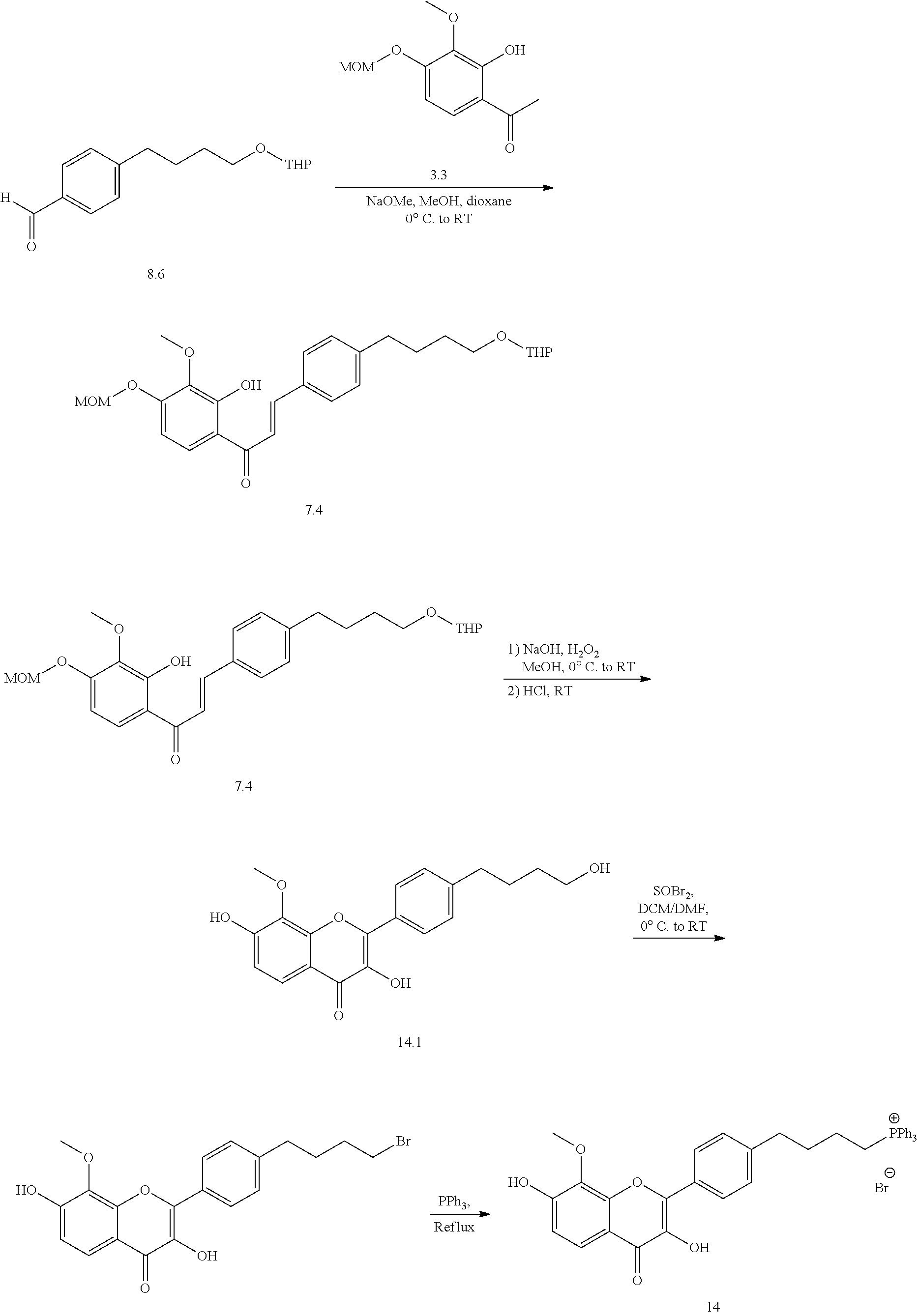

### 3,7-Dihydroxy-2-(4-(4-hydroxybutyl)phenyl)-8-methoxy-4H-chromen-4-one (14.1)

An aqueous solution of hydrogen peroxide (2.828 g, 2.547 mL, 35% Wt, 2.20 Eq, 29.10 mmol) was added to an ice-cold suspension of 7.4 (6.224 g, 1.00 Eq, 13.23 mmol) in methanol (75 mL) and sodium hydroxide (3.5 g, 2.7 mL, 30% Wt, 2.00 Eq, 26.45 mmol). The reaction had reached completion after 18 hours. Then the reaction mixture was cooled in an ice-bath and distilled water (15 mL) was added, followed by a small amount of saturated aqueous citric acid (until the aqueous layer becomes neutral or slightly acidic). More water was added (50 mL). The mixture was extracted with dichloromethane (3×60 mL). The combined organic layers were washed with brine (30 mL). The brine layer was further extracted with dichloromethane (20 mL). The organic layers were dried over Na₂SO₄ and concentrated under reduced pressure to give a brown solid (3.573 g, 56% yield, 7.374 mmol). The residue was dissolved in dioxane (25 mL), cooled in an ice-bath and treated with HCl (13.50 g, 92.59 mL, 4 molar, 28 Eq, 370.4 mmol). The reaction was done within 60 minutes and the solvent was removed by evaporation to yield a brown oil (3.542 g). This gave a mixture of partially THP protected/unprotected product.

### 2-(4-(4-Bromobutyl)phenyl)-3,7-dihydroxy-8-methoxy-4H7-chromen-4-one (14.2)

A solution of 14.1 (2.708 g, 1 Eq, ˜7.598 mmol) in dry DCM (20 mL) and DMF (9 mL) was cooled to 0° C. under nitrogen atmosphere. Then, thionyl bromide (3.2 g, 1.2 mL, 2.7 Eq, 15 mmol) was added. The reaction had reached completion within 2 h. Upon completion, the reaction mixture was cooled with ice-bath and 80 mL of sat. NaHCO₃ were added. The mixture was then extracted with 3×100 mL of DCM. Organic layers were combined and dried with sodium sulfate. The solution was filtered and concentrated, yielding crude product (combined here with an identical reaction on a smaller scale—0.15 g). This was purified by column chromatography (DCM:MeOH gradient), yielding 14.2 (2.499 g, 5.96 mmol, 45% yield from 7.4, 91% purity) as a pale yellow solid. Later, part of 14.2 (1.83 g) was purified by normal phase chromatography again to give 14.2 (1.154 g, 2.752 mmol 92% purity).

(4-(4-(3,7-Dihydroxy-8-methoxy-4-oxo-4H1-chromen-2-yl)phenyl)butyl)triphenylphosphonium bromide (14).

1^{St} Batch:

14.2 (0.404 g, 1.00 Eq, 964 μmol) was dissolved in dry dioxane (3 mL) in a microwave vial, by heating. Then triphenylphosphine (1.26 g, 5 Eq, 4.82 mmol) and sodium iodide (7.22 mg, 0.05 Eq, 48.2 μmol) were added, the reaction purged with N₂. The additional reagents dissolved by sonication. The vial was then heated at 100° C. for 2 days. The reaction was cooled and precipitated with toluene (6 mL). The precipitate was filtered, washed with toluene (3×4 mL) and Et₂O (3×4 mL). Upon drying, TPPO was still present in the precipitate so it was triturated further with both toluene and Et₂O. This gave a yellow solid (643 mg). This was purified by column chromatography to give the product as a pure 14 as a yellow powder (567 mg, 832 μmol, 86% yield, 95.4% purity)

2^{Nd} Batch:

14.2 (0.231 g, 1.00 Eq, 551 μmol) was dissolved in dry dioxane (1 mL) in a microwave vial, by heating. Then triphenylphosphine (723 mg, 5 Eq, 2.75 mmol) and sodium iodide (4.13 mg, 0.05 Eq, 27.5 μmol) were added, the reaction purged with N₂. The additional reagents were dissolved by sonication. The vial was then heated at 105° C. for 23 hours. The reaction was cooled and precipitated with toluene (6 mL). The precipitate was filtered, washed with toluene (3×3 mL) and Et₂O (3×3 mL). The solid was dissolved in methanol, and the solvent removed under reduced pressure to give a yellow solid (409 mg). This was purified by column chromatography to give the product as a pure 14 as a yellow powder (310 mg, 455 μmol, 83% yield, 98.9% purity).

### Synthesis Example 2: SND 200

The Following Route of Synthesis was Adopted to Prepare SND 20 (Compound 17.5):

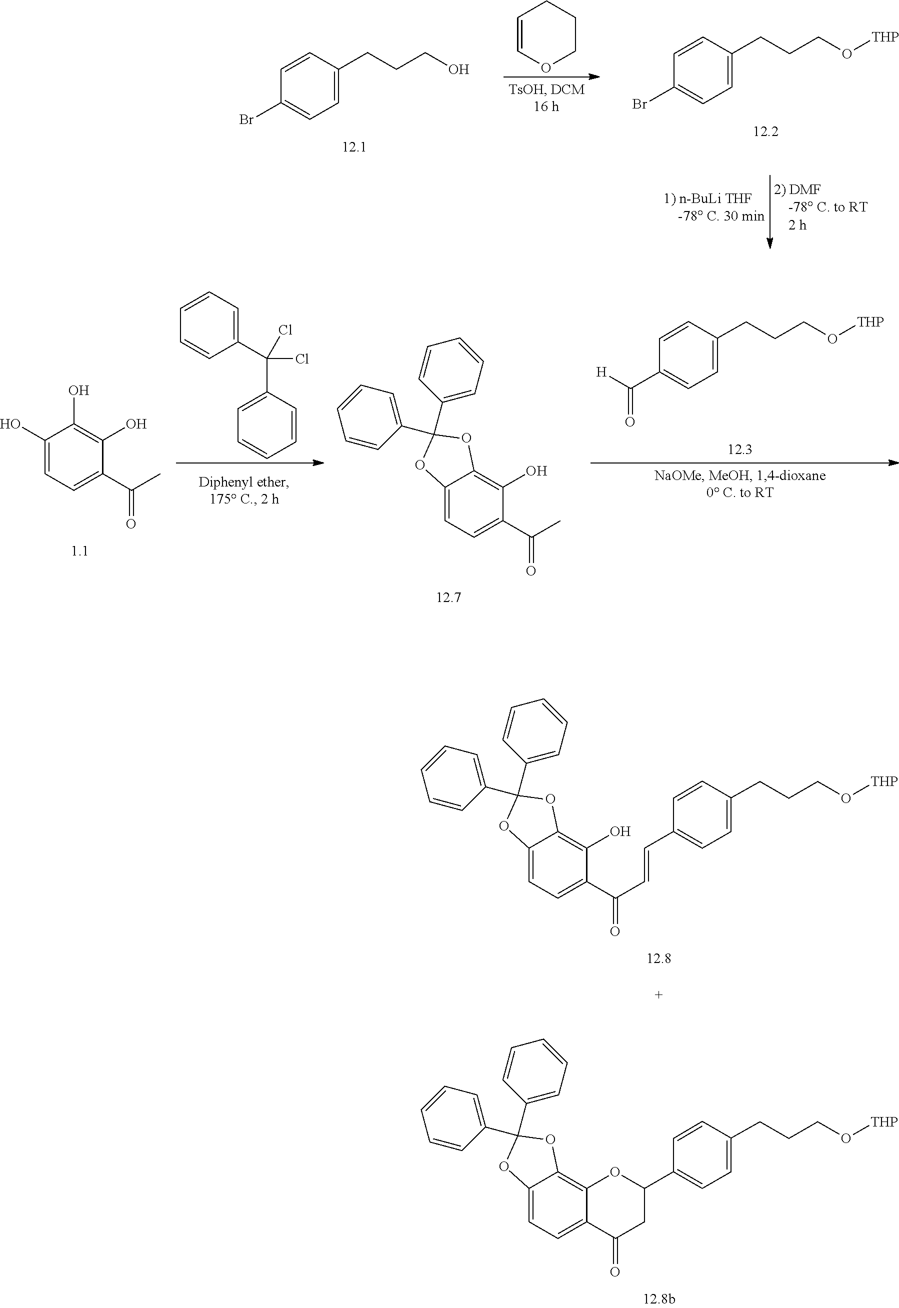

### 2-(3-(4-Bromophenyl)propoxy)tetrahydro-2H-pyran (12.2)

A solution of 3-(4-bromophenyl)propan-1-ol (12.1) (24.97 g, 1 Eq, 116.1 mmol) in dichloromethane (250 mL) was cooled under gentle nitrogen flow to 0° C. in a 500 ml round-bottom flask. p-Toluenesulfonic acid monohydrate (2.21 g, 0.111 Eq, 12.8 mmol) was then added portion-wise. 3,4-Dihydro-2H-pyran (19.35 g, 1.981 Eq, 230.0 mmol) was added drop-wise from a dropping funnel within 30 min before the mixture was allowed to warm to room-temperature. The solution turned eventually to black. The reaction mixture was stirred at room temperature for 16 hours before it was concentrated. The resultant black oil was purified by flash-chromatography using ethyl acetate/heptanes to yield 2-(3-(4-bromophenyl)propoxy)tetrahydro-2H-pyran (12.2) (28.8 g, 96.3 mmol, 83%, 100% purity) as a transparent oil.

### 4-(3-((Tetrahydro-2H-pyran-2-yl)oxy)propyl)benzaldehyde (12.3)

2-(3-(4-Bromophenyl)propoxy)tetrahydro-2H-pyran (12.2, 27.67 g, 1 Eq, 92.48 mmol) and THF (310 mL) were transferred under nitrogen flow to a flame-dried 500 ml three-neck round-bottom flask. The solution was cooled under gentle nitrogen flow to −75° C., before n-butyllithium (6.49 g, 40.5 mL, 2.5 molar, 1.09 Eq, 101 mmol) in hexanes was added portion-wise within 20 min. After 30 min of stirring, dry DMF was added portion-wise within 25 min and the reaction mixture was stirred for another 5 min before the cooling bath was removed. The reaction mixture was then stirred at 20° C. for 2 hour, before the reaction mixture was quenched with 100 ml of water and diluted with 900 ml of water. Resulting suspension was extracted with 3×750 ml of EtOAc. The organic fractions were combined, dried with sodium sulfate, filtered and concentrated to give the crude product as a yellow oil. The crude product was purified by flash-chromatography using ethyl acetate/heptanes to yield 4-(3-((tetrahydro-2H-pyran-2-yl)oxy)propyl)benzaldehyde (12.3) (18.7 g, 75 mmol, 81%, 99% purity) as a colorless oil.

### 1-(4-Hydroxy-2,2-diphenylbenzo[d][1,3]dioxol-5-yl)ethan-1-one (12.7)

1-(2,3,4-Trihydroxyphenyl)ethan-1-one (10.86 g, 1 Eq, 64.59 mmol), dichlorodiphenylmethane (15.29 g, 12.38 mL, 1.00 Eq, 64.48 mmol) and diphenyl ether (85 mL) were transferred under nitrogen flow to a 250 ml three-neck flask. The reaction mixture was heated at 175° C. for 30 min. The reaction mixture was allowed to cool to room temperature before it was poured to 900 ml of heptane. After a couple of minutes, precipitate started to form. This was filtered and washed with heptane. The dark precipitate on the filter was dissolved in DCM, 25 mL of EtOAc and 25 mL of heptane was added. This mixture was then concentrated until extensive precipitate formed. This was filtered, washed with 4×25 mL of EtOAc:heptane 1:1 mixture and purified by normal phase flash-chromatography using EtOAc:heptane as the eluent. The filtrate of the first filtration was concentrated, cooled to 4° C. for 20 h, filtered and washed with heptane This was combined with the material recovered from flash-chromatography to yield 1-(4-hydroxy-2,2-diphenylbenzo[d][1,3]dioxol-5-yl)ethan-1-one (12.7) (15.62 g, 47.0 mmol, 73%, 100% purity) as a white solid.

(E)-1-(4-Hydroxy-2,2-diphenylbenzo[d][1,3]dioxol-5-yl)-3-(4-(3-((tetrahydro-2H-pyran-2-yl)oxy)propyl)phenyl)prop-2-en-1-one (12.8) and 2,2-diphenyl-8-(4-(3-((tetrahydro-2H-pyran-2-yl)oxy)propyl)phenyl)-7,8-dihydro-6H-[1,3]dioxolo[4,5-h]chromen-6-one (12.8b).

Sodium methoxide (37.9 g, 130 mL, 5.4 molar, 35.7 Eq, 702 mmol) in MeOH was added portion-wise under nitrogen flow to an ice/NaCl cooled suspension of 1-(4-hydroxy-2,2-diphenylbenzo[d][1,3]dioxol-5-yl)ethan-1-one (6.5287 g, 1 Eq, 19.643 mmol) and 4-(3-((tetrahydro-2H-pyran-2-yl)oxy)propyl)benzaldehyde (5.037 g, 1.033 Eq, 20.28 mmol) in 1,4-dioxane (70 mL) at 0° C. The mixture was allowed slowly to warm to room temperature and it was stirred for 15 h under nitrogen atmosphere. The reaction mixture was then poured to 500 ml of ice-cold brine. The resultant suspension was extracted with 3×100 ml of EtOAc. Organic fractions were combined, dried with sodium sulfate, filtered and evaporated to dryness, yielding 14.27 g of dark orange oil. The crude product was suspended in DCM and purified twice by normal phase flash-chromatography using DCM:MeOH as the eluent, to yield (E)-1-(4-hydroxy-2,2-diphenylbenzo[d][1,3]dioxol-5-yl)-3-(4-(3-((tetrahydro-2H-pyran-2-yl)oxy)propyl)phenyl)prop-2-en-1-one (12.8) (5.16 g, 9.17 mmol, 46.7%, 100% purity) as an orange foam and 2,2-diphenyl-8-(4-(3-((tetrahydro-2H-pyran-2-yl)oxy)propyl)phenyl)-7,8-dihydro-6H-[1,3]dioxolo[4,5-h]chromen-6-one (12.8b) (4.825 g, 7.7 mmol, 39%, 90% purity) a yellow foam.

### 7-Hydroxy-2,2-diphenyl-8-(4-(3-((tetrahydro-2H-pyran-2-yl)oxy)propyl)phenyl)-6H-[1,3]dioxolo[4,5-h]chromen-6-one (17.6)

(E)-1-(4-Hydroxy-2,2-diphenylbenzo[d][1,3]dioxol-5-yl)-3-(4-(3-((tetrahydro-2H-pyran-2-yl)oxy)propyl)-phenyl)prop-2-en-1-one (12.8) (3.527 g, 1 Eq, 6.268 mmol) was dissolved in a mixture of MeOH (37 mL) and sodium hydroxide in water (1.65 g, 1.25 mL, 30% Wt, 1.97 Eq, 12.4 mmol). The resultant red solution was stirred for 10 min at room temperature before hydrogen peroxide (1.4 g, 1.2 mL, 35% Wt, 2.2 Eq, 14 mmol) was added at 0° C. The mixture was stirred at 0° C. for 10 min before it was allowed to warm to room temperature and stirred at room temperature for 18 h. The reaction mixture was then cooled to 0° C. and 70 mL of water was added. The resultant yellow suspension was acidified with 10% of citric acid until pH was between 2 and 4. The aqueous layer was extracted with 2×200 mL of DCM, organic layers were combined and evaporated to dryness. Crude product was purified by normal phase flash-chromatography using EtOAc:heptanes. 7-Hydroxy-2,2-diphenyl-8-(4-(3-((tetrahydro-2H-pyran-2-yl)oxy-propyl)phenyl)-6H-[1,3]dioxolo[4,5-h]chromen-6-one (17.6) (1.651 g, 2.863 mmol, 41%, 90% purity) was obtained as a beige powder.

### 8-(4-(3-Bromopropyl)phenyl)-7-hydroxy-2,2-diphenyl-6H-[1,3]dioxolo[4,5-h]chromen-6-one (17.7)

Thionyl bromide (1.5 g, 0.55 mL, 2.5 Eq, 7.1 mmol) was added to a solution of 7-hydroxy-2,2-diphenyl-8-(4-(3-((tetrahydro-2H-pyran-2-yl)oxy)propyl)phenyl)-6H-[1,3]dioxolo[4,5-h]chromen-6-one (17.6) (1.65 g, 1 Eq, 2.86 mmol) and dry DMF (2.2 g, 2.3 mL, 10 Eq, 30 mmol) in dry DCM (23 mL) at 0° C. After 5 min at 0° C., cooling bath was removed and the solution was stirred at room temperature for 1.5 h. The reaction mixture was cooled with an ice-bath before it was quenched with 70 mL of sat. aq. NaHCO₃. The mixture was then extracted with 2×100 ml of DCM. Organic layers were combined and evaporated to dryness. Crude product was purified by normal phase flash-chromatography using EtOAc:heptanes. 8-(4-(3-Bromopropyl)phenyl)-7-hydroxy-2,2-diphenyl-6H-[1,3]dioxolo[4,5-h]chromen-6-one (17.7) (1.398 g, 2.3 mmol, 79%, 90% purity) was obtained as a light brown solid.

(3-(4-(7-Hydroxy-6-oxo-2,2-diphenyl-6H-[1,3]dioxolo[4,5-h]chromen-8-yl)phenyl)propyl)triphenylphosphonium bromide (17.9).

Triphenylphosphine (354 mg, 5 Eq, 1.35 mmol) was added to a mixture of 8-(4-(3-bromopropyl)phenyl)-7-hydroxy-2,2-diphenyl-6H-[1,3]dioxolo[4,5-h]chromen-6-one (17.7) (0.150 g, 1 Eq, 270 μmol) and sodium iodide (6.07 mg, 0.15 Eq, 40.5 μmol) in 1,4-dioxane (3 mL). The reaction mixture was refluxed for 18 h under nitrogen atmosphere, before it was allowed to cool to room temperature. The resultant suspension was diluted with 5 mL of toluene and filtered. The white solid on the filter was washed with 3×5 mL of toluene and 3×3 mL of water. After drying, (3-(4-(7-hydroxy-6-oxo-2,2-diphenyl-6H-[1,3]dioxolo[4,5-h]chromen-8-yl)phenyl)propyl)-triphenylphosphonium bromide (0.142 g, 174 μmol, 63%, 98% purity) was obtained as a white solid.

(3-(4-(7,8-Dihydroxy-4-oxo-4H1-chromen-2-yl)phenyl)propyl)triphenylphosphonium bromide (17.5).

(3-(4-(6-Oxo-2,2-diphenyl-6H-[1,3]dioxolo[4,5-h]chromen-8-yl)phenyl)propyl)triphenylphosphonium bromide (17.9) (122 mg, 1 Eq, 152 μmol) was suspended in MeCN (0.5 mL) and deprotected with c. HBr (641 mg, 433 μL, 48% Wt, Eq, 3.80 mmol) by using the general method for deprotection. (3-(4-(7,8-Dihydroxy-4-oxo-4H-chromen-2-yl)phenyl)propyl)triphenylphosphonium bromide (17.5) 79 mg, 0.12 mmol, 78%, 96% purity) was obtained as an orange powder.

### Examples—Biological Studies

Experimental Methodology

Antitumor activity of the compounds and doxorubicin as a positive control was assessed by using the CellTiter-Blue Cell Viability Assay (Promega, #G8082) or CellTiter-Glow® Luminescent Cell Viability assay (Promega #G7572) according to the manufacturer's instructions. The compounds were tested at 5 or 6 concentrations in half-log increments (highest concentration 30 μM or 100 μM) in duplicate or triplicate well conditions.

Tumor cells were grown at 37° C. in a humidified atmosphere with 5% CO₂ in RPMI 1640 or DMEM medium, supplemented with 10% (v/v) fetal calf serum and 50 μg/ml gentamicin for up to 20 passages, and were passaged once or twice weekly. Cells were harvested using TrypLE or PBS buffer containing 1 mM EDTA, and the percentage of viable cells was determined using a CASY Model TI cell counter (OMNI Life Science). Cells were harvested from exponential phase cultures, counted and plated in 96 well flat-bottom microtiter plates at a cell density depending on the cell line's growth rate (4,000-20,000 cells/well depending on the cell line's growth rate, up to 60,000 for hematological cancer cell lines) in RPMI 1640 or DMEM medium supplemented with 10% (v/v) fetal calf serum and 50 μg/ml gentamicin (140 μl/well). Cultures were incubated at 37° C. and 5% CO₂ in a humidified atmosphere. After 24 h, 10 μl of test compounds or control medium were added and left on the cells for another 72 h. Compounds were serially diluted in DMSO, transferred in cell culture medium, and added to the assay plates. The DMSO concentration was kept constant at <0.3% v/v across the assay plate. Viability of cells was quantified by the CellTiter-Blue® cell viability assay (Promega G8081) or CellTiter-Glow® Luminescent Cell Viability assay (Promega #G7572). Fluorescence (FU) was measured by using the EnSpire® multimode plate reader (Perkin Elmer) (excitation λ=570 nm, emission λ=600 nm) Luminescence was measured with a microplate luminometer (Promega or PerkinElmer).

Sigmoidal concentration-response curves were fitted to the data points (test-versus-control, T/C values) obtained for each tumor model using 4 parameter non-linear curve fit (Charles River DRS Datawarehouse Software) or with GraphPad prism 5.02 software. IC₅₀ values are reported as absolute IC₅₀ values, being the concentration of test compound at the intersection of the concentration-response curves with T/C=50% Cell lines tested are presented in Table 1.

TABLE 1
Tumour cell lines type and designation

| Tumour model | Cell line |
|---|---|
| Brain | U-87 |
| | SF-268 |
| | U-251 |
| | IMR-5-75 |
| | SK-N-SH |
| | Kelly |
| | SK-N-AS |
| | SH-SY-5Y |
| | CHP-134 |
| | U-118 |
| Breast | MCF-7 |
| | MDA-MB-468 |
| | BT-747 |
| | MDA-MB-436 |
| | MDA-MB-231 |
| | HCC1806 |
| | ZR-75-1 |
| | T47D |
| Colon | HC-T116 |
| | HT-29 |
| | HCT-15 |
| | LoVo |
| | KM-12 |
| Leukemia | K-562 |
| | HL-60 |
| Lung (NSCLC) | A-549 |
| | H-1299 |
| | Calu-6 |
| | NCI-H460 |
| Lung (SCLC) | H69AR |
| | NCI-H69 |
| | DMS-114 |
| Lymphoma | U937 |
| | Farage |
| Ovarian | SK-OV-3 |
| | OVCAR-3 |
| | 899 |
| Pancreatic | Mia-Pa-Ca-2 |
| | BxPC-3 |
| | Panc-1 |
| Prostate | PC-3 |
| | LNCaP |
| | 22Rv1 |
| Renal | 486L |
| Skin (Melanoma) | A375 |
| | SK-Mel-28 |
| | SK-Mel-5 |
| | A2058 |
| | MeWo |

Antitumor Activity Against a Panel of Patient-Derived Xenografts (PDX)

PDX-derived cell cultures were obtained from tumors explanted from mice and isolated by mechanical and enzymatic dissociation. Assays were performed on cells from frozen stocks at least 2 weeks after thawing and maintained in culture at 37° C. in a humidified atmosphere with 5% CO₂ in complete growth medium supplemented with 8 to 16% fetal bovine serum, 1% Penicillin-Streptomycin (10,000 U/mL), 2 mM L-Glutamine+/−Insulin-Transferrin-Selenium 1X and Albumax II (10 to 40 μM depending on cell type). Cells were harvested and seeded in 96-wells plates at a density of 1.25 to 5×10³ cells/well for cytotoxicity assays. Cells were incubated 48 h at 37° C. prior to addition of test molecules and vehicle (DMSO, 0.1%) at desired final concentrations. Cell viability was assessed before drugs' addition (To) and 5 days after test molecules addition by measuring ATP cell content using CellTiter-Glo® Luminescent Cell Viability Assay (Promega) according to the manufacturer's instructions. Luciferase activity was measured on a luminometer (PerkinElmer® EnVision™). Each concentration of compounds was tested in triplicate.

Viability was calculated as a percentage of ATP value compared to vehicle treated controls.

For PDX primary cell cultures, the tumour tissue was washed with PBS containing antibiotic-antimycotic and non-tumour tissue and necrotic tumour tissues were separated. The tumour tissue was transferred to a new dish and cut into 1˜2 mm³ fragments, resuspended in RPMI-1640 medium and centrifuged at 1,200 rpm for 6 min at room temperature. The pelleted material was resuspended with 15 mL of Tumour Cell Digestion Solution and incubated at 37° C. for 1 hour with agitation. Following further addition of media, centrifugation and passage through a 70 μm cell strainer, the homogenous cell mixture was layered onto 15 mL of Ficoll-Paque PLUS in a 50 mL conical tube and centrifuged for 15 min at 1,600 rpm. The interface cells were collected, washed with media, separated by centrifugation at 1,200 rpm. The cell pellet was resuspended in serum free media supplemented with growth factors. 10,000 cells/wells were plated in a 96 well plate and incubated at 37° C., 5% CO₂, 95% air and 100% relative humidity overnight. The cytotoxicity assay was conducted as above.

Sigmoidal concentration-response curves were fitted to the data points (test-versus-control, T/C values) obtained for each tumor model using GraphPad prism 5.02 software. IC₅₀ values are reported as absolute IC₅₀ values, PDX tested are presented in Table 2.

TABLE 2
PDX origin and designation

| Tumour organ | Model ID |
|---|---|
| Bile duct | CH-17-0091 |
| | CH-17-0098 |
| Brain | GBM14-CHA |
| | ODA14-RAV |
| Breast | HBCx-2 |
| | HBCx-3 |
| | HBCx-6 |
| | BR-05-0300 |
| | BR-05-0014E |
| Colon | TC71 |
| | CO-04-0722 |
| | CO-04-0701 |
| | CO-04-0700 |
| Endometrium | END4-HIR |
| | EN11-01-01 |
| Esophagus | ES-06-0002 |
| | ES-06-0122 |
| Head and neck | HN-13-0020 |
| Kindney | Ki-12-0062 |
| Liver | HB-214-FOI |
| Lung | IC20-DAN |
| | SC6 |
| | LU-01-0027 |
| | LU-01-0010 |
| | LU-01-0604 |
| | LU-01-0025 |
| Lymphoma | LY-24-0304 |
| Ovary | OVA2-BUR |
| Pancreas | PC-07-0045 |
| | PC-07-0059 |
| Prostate | HID28 |
| Skin | MCM002-FJ |
| | ME-21-0028 |
| Stomach | ST-02-0007 |
| | ST-02-0173 |
| | ST-02-0012 |
| | ST-02-0322 |

Inhibition of Kinase Activity

Selected compounds were screened for kinase inhibition using the KINOMEscan™ assay (Eurofins) which is based on a competition binding assay that quantitatively measures the ability of a compound to compete with an immobilized, active-site directed ligand. The assay was performed by combining three components: DNA-tagged kinase; immobilized ligand; and the test compound. The ability of the test compound to compete with the immobilized ligand was measured via quantitative PCR of the DNA tag.

Kinase-tagged T7 phage strains were prepared in an E. coli host derived from the BL21 strain. E. coli were grown to log-phase and infected with T7 phage and incubated with shaking at 32° C. until lysis. The lysates were centrifuged and filtered to remove cell debris. The remaining kinases were produced in HEK-293 cells and subsequently tagged with DNA for qPCR detection. Streptavidin-coated magnetic beads were treated with biotinylated small molecule ligands for 30 minutes at room temperature to generate affinity resins for kinase assays. The liganded beads were blocked with excess biotin and washed with blocking buffer (SeaBlock (Pierce), 1% BSA, 0.05% Tween 20, 1 mM DTT) to remove unbound ligand and to reduce non-specific binding. Binding reactions were assembled by combining kinases, liganded affinity beads, and test compounds in 1×binding buffer (20% SeaBlock, 0.17×PBS, 0.05% Tween 20, 6 mM DTT). Test compounds were prepared as 111X stocks in 100% DMSO. Kds were determined using an 11-point 3-fold compound dilution series with three DMSO control points. All compounds for Kd measurements were distributed by acoustic transfer (non-contact dispensing) in 100% DMSO. The compounds were then diluted directly into the assays such that the final concentration of DMSO was 0.9%. All reactions performed in polypropylene 384-well plate. Each was a final volume of 0.02 ml. The assay plates were incubated at room temperature with shaking for 1 hour and the affinity beads were washed with wash buffer (1×PBS, 0.05% Tween 20). The beads were then re-suspended in elution buffer (1×PBS, 0.05% Tween 20, 0.5 μM nonbiotinylated affinity ligand) and incubated at room temperature with shaking for 30 minutes. The kinase concentration in the eluates was measured by qPCR. Compounds were initially tested at a concentration of 10 mM against a panel of 30 kinases and results for primary screen binding interactions were reported as ‘% Ctrl’

% Ctrl Calculation=(test compound signal−positive control signal/negative control signal−positive control signal)×100, where negative control=DMSO (100% Ctrl) positive control=control compound (0% Ctrl). Test compounds with % Ctrl between 0 and 10 were selected for Kd determination.

Binding constants (Kds) were calculated with a standard dose-response curve using the Hill equation: Response=Background+[Signal−Background/1+(Kd^{Hill Slope}/Dose^{Hill Slope})]

The Hill Slope was set to −1. Curves were fitted using a non-linear least square fit with the Levenberg-Marquardt algorithm.

### Example 1. Activity Against Bile Duct Patient-Derived Xenografts (PDX)

SND190 and SND200 inhibited bile duct PDX growth with IC₅₀ below 10 μM as presented in Table 3.

TABLE 3
IC50 values against bile duct carcinomas

| PDX/IC50 (μM) | CH-17-0091 | CH-17-0098 |
|---|---|---|
| SND190 | 4 | 4.0 |
| SND200 | 9.5 | 7.5 |

### Example 2. Activity Against Brain Carcinoma Cell Lines

SND190 and SND200 inhibited brain cancer cell growth with IC₅₀ s below 20 μM, as presented in Tables 3A-3C.

TABLE 3A
IC50 values against brain carcinoma cell lines

| Cell Line/IC50 | |
|---|---|
| (μM) | SF-268 |
| SND190 | 4.10 |
| SND200 | 7.83 |

TABLE 3B
IC50 values against brain carcinoma cell lines

| Cell Line/IC50 | |
|---|---|
| (μM) | U-251 |
| SND190 | 7.54 |

TABLE 3C
IC50 values against brain carcinoma cell lines

| Cell Line/IC50 | | | | |
|---|---|---|---|---|
| (μM) | SK-N-AS | SH-SY-5Y | CHP-134 | U-118 |
| SND190 | 3 | 2.6 | 1.1 | 9.4 |

### Example 3. Activity Against Breast Carcinoma

SND190 and SND200 inhibited breast cancer cell growth with IC₅₀ s below 5 μM, as presented in Tables 4A & 4B.

TABLE 4A
IC50 values against breast carcinoma cell lines

| Cell Line/IC50 | | |
|---|---|---|
| (μM) | MCF-7 | MDA-MB-468 |
| SND190 | 3.91 | 0.3 |
| SND200 | 1.68 | 0.3 |

TABLE 4B
IC₅₀ values against breast carcinoma cell lines

| Cell Line/ | BT- | MDA-MB- | MDA-MB- | | ZR- | |
|---|---|---|---|---|---|---|
| IC₅₀ (μM) | 474 | 436 | 231 | HCC1806 | 75-1 | T47D |
| SND190 | 1.9 | 2.9 | 3.5 | 2.8 | 3.5 | 1.3 |

SND190 and SND200 inhibited the growth of breast PDX with IC₅₀ s below 20 μM as shown in Table 5.

TABLE 5
IC50 values against breast PDX

| PDX/IC50 (μM) | BR-05-0399 | BR-05-0014E |
|---|---|---|
| SND190 | 5.4 | 6.6 |
| SND200 | 14.9 | 19 |

### Example 4. Activity Against Colon Carcinoma

SND190 and SND200 inhibited colon cancer cell growth with IC₅₀ s below 20 μM, as presented in Table 6.

TABLE 6
IC50 values against colon carcinoma cell lines

| Cell Line/IC50 | | | | | |
|---|---|---|---|---|---|
| (μM) | HCT-116 | LoVo | HT-29 | HCT-15 | KM-12 |
| SND190 | 0.81 | 2.49 | 5.78 | 14.96 | 1.7 |
| SND200 | 1.48 | 0.64 | 3.31 | 7.49 | 0.44 |

SND190 and SND200 inhibited the growth of colon PDX with IC₅₀ s below 20 μM as shown in Table 7

TABLE 7
IC50 values against colon PDX

| PDX/IC50 (μM) | CO-04-0722 | CO-04-0701 | CO-04-0700 |
|---|---|---|---|
| SND190 | 4.4 | 14.7 | 2.4 |
| SND200 | 9.8 | 15.1 | 7.6 |

### Example 5. Activity Against Endometrial Cancer

SND190 and SND200 inhibited the endometrial PDX growth with IC₅₀ s below 20 μM as presented in Table 8.

TABLE 8
IC50 values against endometrial PDX

| PDX/IC50 | |
|---|---|
| (μM) | EN-11-0101 |
| SND190 | 7.9 |
| SND200 | 10.4 |

### Example 6. Activity Against Esophagus PDX

SND190 and SND200 inhibited the esophagus PDX growth with IC₅₀ s below 20 μM as presented in Table 9.

TABLE 9
IC₅₀ values against esophagus PDX

| PDX/IC₅₀ | | |
|---|---|---|
| (μM) | ES-06-0002 | ES-06-0122 |
| SND190 | 4.9 | 6.5 |
| SND200 | 11.6 | 8.8 |

### Example 7. Activity Against Head and Neck Cancer

SND190 and SND200 inhibited the head and neck PDX growth with IC₅₀ s below 5 μM as presented in Table 10.

TABLE 10
IC₅₀ values against head and neck PDX

| PDX/IC₅₀ (μM) | HN-13-0020 |
|---|---|
| SND190 | 2.26 |
| SND200 | 4.1 |

### Example 8. Activity Against Kidney Cancer

SND190 and SND200 inhibited the head and neck PDX growth with IC₅₀ s below 20 M as presented in Table 11.

TABLE 11
IC₅₀ values against kidney PDX

| PDX/IC₅₀ (μM) | KI-12-0062 |
|---|---|
| SND190 | 15.1 |
| SND200 | 14.5 |

### Example 9. Activity Against Leukaemia

SND190 and SND200 inhibited leukaemia cell growth with IC₅₀ s below 1 μM, as presented in Table 12.

TABLE 12
IC₅₀ values against leukaemia cell lines

| Cell Line/IC₅₀ | |
|---|---|
| (μM) | HL-60 |
| SND190 | 0.3 |
| SND200 | 0.3 |

### Example 10 Activity Against Lung Carcinoma

SND190 inhibited lung carcinoma cell growth with IC₅₀ s below 20 μM, as presented in Table 13.

TABLE 13
IC₅₀ values against lung carcinoma cell lines

| Cell Line/IC₅₀ | | | |
|---|---|---|---|
| (μM) | CALU-6 | NCI-H460 | A549 |
| SND190 | 2.39 | 8.63 | 13.83 |

SND200 inhibited growth of the SCLC doxorubicin resistant cell line H69AR, as depicted in Table 14. SND190 exhibited a good potency against the parental H69 cells with IC₅₀ of 1.42 μM.

TABLE 14
IC₅₀ values against resistant SCLC

| Cell Line/IC₅₀ | |
|---|---|
| (μM) | SND200 |
| H69AR | 4-25 |

SND190 and SND200 inhibited the lung PDX growth with IC₅₀ s below 20 μM as presented in Table 15.

TABLE 15
IC₅₀ values against lung PDX

| PDX/IC₅₀ | LU-01- | LU-01- | LU-01- | LU-01- | LU-01- |
|---|---|---|---|---|---|
| (μM) | 0027 | 0010 | 0604 | 0004 | 0025 |
| SND190 | 4 | 1.1 | 2 | 5.4 | 0.6 |
| SND200 | 1.8 | 10 | 7.8 | 9.8 | 4.9 |

### Example 11. Activity Against Lymphoma

SND190 and SND200 inhibited the lymphoma PDX growth with IC₅₀ s below 5 μM as presented in Table 16.

TABLE 16
IC₅₀ values against lymphoma PDX

| PDX/IC₅₀ (μM) | LY-24-0340 |
|---|---|
| SND190 | 3.8 |
| SND200 | 4 |

### Example 12. Activity Against Ovarian Carcinoma

SND190 and SND200 inhibited ovarian cancer cell growth with IC₅₀ s below 20 μM, as presented in Table 17.

TABLE 17
IC₅₀ values against ovarian carcinoma

| Cell Lin/IC₅₀ | | |
|---|---|---|
| (μM) | OVXF 899 | OVCAR-3 |
| SND190 | 8.76 | 3.81 |
| SND200 | 6.37 | 1.6 |

### Example 13. Activity Against Pancreatic Carcinoma

SND190 and SND200 inhibited pancreatic cancer cell growth with IC₅₀ s below 20 μM, as presented in Table 18A and 18B

TABLE 18A
IC₅₀ values against pancreatic carcinoma

| Cell Line/IC₅₀ | Mia-Pa-Ca- |
|---|---|
| (μM) | 2 |
| SND190 | 0.33 |

SND200 inhibit Panc-1 cell line as presented in Table 18B.

TABLE 18B
IC₅₀ values against pancreatic carcinoma

| Cell Line/IC₅₀ | |
|---|---|
| (μM) | Panc-1 |
| SND200 | 1.33 |

SND190 and SND200 inhibited the pancreatic PDX growth with IC₅₀ s below 10 μM as presented in Table 19.

TABLE 19
IC₅₀ values against pancreatic PDX

| PDX/IC₅₀ (μM) | PC-07-0045 | PC-07-0059 |
|---|---|---|
| SND190 | 3.4 | 2.3 |
| SND200 | 2.3 | 6.7 |

### Example 14. Activity Against Prostate Carcinoma

SND190 and SND200 inhibited prostate cancer cell growth with IC₅₀ s below 10 μM, as presented in Table 20. The data suggests that the whole class of these novel derivatives is very potent against all prostate cell lines.

TABLE 20
IC₅₀ values against prostate carcinoma

| Cell Line/IC₅₀ | | | |
|---|---|---|---|
| (μM) | PC-3 | ONCap | 22Rv1 |
| SND190 | 1.69 | 1.5 | 0.79 |
| SND200 | 0.3 | 0.52 | 0.42 |

### Example 15. Activity Against Skin Melanoma

SND190 and SND200 inhibited the skin melanoma PDX growth with IC₅₀ s below 20 M as presented in Table 21.

TABLE 21
IC₅₀ values against skin melanoma PDX

| PDX/IC₅₀ (μM) | ME-21-0028 |
|---|---|
| SND190 | 2.2 |
| SND200 | 10.9 |

### Example 16. Activity Against Stomach Cancer

SND190 and SND200 inhibited the stomach PDX growth with IC₅₀ s below 20 μM as presented in Table 22.

TABLE 22
IC₅₀ values against stomach PDX

| PDX/IC₅₀ (μM) | ST-02-0007 | ST-02-0173 | ST-020012 | ST-02-0322 |
|---|---|---|---|---|
| SND190 | 5.7 | 11 | 4.5 | 6.3 |
| SND200 | 9.4 | 15.5 | 8.8 | 8.5 |

### Example 17. Kinase Inhibition Activity

In order to further understand if the tumour inhibition activity is due to the inhibition of certain cancer associated kinases, selected compounds were tested in the KINOMEscan™ assay against 30 kinases. SND190 showed selective inhibitory activity against a small number of kinases as presented in Table 23.

TABLE 23
Kd values kinase inhibition

| | No. | | |
|---|---|---|---|
| | responsive | | |
| Cpd | kinases | Kinase name | Kd (μM) |
| SND190 | 8 | ABL1- | 0.46 |
| | | nonphosphorylated | |
| | | ADCK3 | 0.1 |
| | | CDK11 | 3.6 |
| | | KIT(D816V) | 0.93 |
| | | RIOK2 | 0.71 |
| | | RSK2(Kin.Dom.1-N- | 1.7 |
| | | terminal) | |
| | | TIE2 | 0.04 |
| | | TRKA | 2.3 |

It will be understood that the present invention has been described above byway of example only. The examples are not intended to limit the scope of the invention.

Various modifications and embodiments can be made without departing from the scope and spirit of the invention, which is defined by the following claims only.

1. A compound of formula (I): 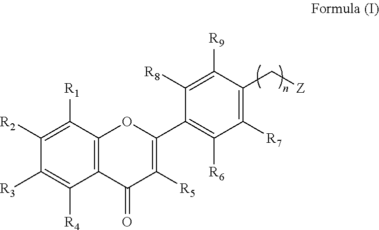
wherein:
Z is —[P(R¹¹)₃]X, wherein X is a counter anion;
R¹, R², and R⁵, independently, are selected from —OH, —O—C₁₋₄ alkyl, —OC(O)R₁₃, —OC(O)NHR¹³, —OC(O)N(R¹³)₂; or
R¹ and R² together form —O—(C₁₋₃ alkylene)-O—; and R⁵ is selected from —OH, —O—C₁₋₄ alkyl, —OC(O)R₁₃, —OC(O)NHR¹³, and —OC(O)N(R¹³)₂;
R³, R⁴, R⁶, R⁷, R⁸, and R⁹, independently, are selected from H; halo; —CN; —NO₂; —R^{β}; —OH, —OR^{β}; —SH; —SR^{β}; —SOR^{β}; —SO₂H; —SO₂R^{β}; —SO₂NH₂; —SO₂NHR^{β}; —SO₂N(R^{β})₂; —NH₂; —NHR^{β}; —N(R^{β})₂; —CHO; —COR^{β}; —COOH; —COOR^{β}; —OCOR^{β}; and benzyl optionally substituted with 1-3—R^{β};
each —R^{β} is independently selected from a C₁-C₆ alkyl, C₂-C₆ alkenyl, C₂-C₆ alkynyl or C₃-C₁₄ cyclic group, and wherein any —R^{β} may optionally be substituted with one or more C₁-C₄ alkyl, C₁-C₄ haloalkyl, C₃-C₇ cycloalkyl, —O(C₁-C₄ alkyl), —O(C₁-C₄ haloalkyl), —O(C₃-C₇ cycloalkyl), halo, —OH, —NH₂, —CN, —NO₂, —C≡CH, —CHO, —CON(CH₃)₂ or oxo (═O) groups;
each —R¹¹ is independently selected from H, C₁-C₆ alkyl, C₂-C₆ alkenyl, C₃-C₁₄ aryl group, or C₃-C₁₄ aliphatic cyclic group, and wherein any —R¹¹ may optionally be substituted with one or more C₁-C₄ alkyl, C₁-C₄ haloalkyl, C₃-C₇ cycloalkyl, —O(C₁-C₄ alkyl), —O(C₁-C₄ haloalkyl), —O(C₃-C₇ cycloalkyl), halo, —OH, —NH₂, —CN, —C≡CH or oxo (═O) groups
each —R¹³ is independently selected from a H, C₁-C₆ alkyl, C₂-C₆ alkenyl, C₂-C₆ alkynyl, or C₃₋₁₄ cyclic group, halo, —NO₂, —CN, —OH, —NH₂, mercapto, formyl, carboxy, carbamoyl, C₁₋₆ alkoxy, C₁₋₆ alkylthio, —NH(C₁₋₆ alkyl), —N(C₁₋₆ alkyl)₂, C₁₋₆ alkylsulfinyl, C₁₋₆ alkylsulfonyl, or arylsulfonyl, wherein any —R¹³ may optionally be substituted with one or more —R¹⁴;
each R¹⁴ is independently selected from a C₁-C₆ alkyl, C₂-C₆ alkenyl, C₂-C₆ alkynyl, C₃₋₁₄ cyclic group, halo, —NO₂, —CN, —OH, —NH₂, mercapto, formyl, carboxy, carbamoyl, C₁₋₆ alkoxy, C₁₋₆ alkylthio, —NH(C₁₋₆ alkyl), —N(C₁₋₆ alkyl)₂, C₁₋₆ alkylsulfinyl, C₁₋₆ alkylsulfonyl, or arylsulfonyl, wherein any —R₁₄ may optionally be substituted with one or more —R₁₅;
each —R¹⁵ is independently selected from halogen, nitro, cyano, hydroxy, trifluoromethoxy, trifluoromethyl, amino, formyl, carboxy, carbamoyl, mercapto, sulfamoyl, methyl, ethyl, methoxy, ethoxy, acetyl, acetoxy, methylamino, ethylamino, dimethylamino, diethylamino, N-methyl-N-ethylamino, acetylamino, N-methylcarbamoyl N-ethylcarbamoyl N,N-dimethylcarbamoyl, N,N-diethylcarbamoyl, N-methyl-N-ethylcarbamoyl, methylthio, ethylthio, methylsulfinyl, ethylsulfinyl, mesyl ethylsulfonyl, methoxycarbonyl, ethoxycarbonyl, N-methylsulfamoyl N-ethylsulfamoyl N,N-dimethylsulfamoyl N,N-diethylsulfamoyl, N-methyl-N-ethylsulfamoyl, carbocyclyl, aryl, or heterocyclyl; and
n=1-10.

2. A compound as claimed in claim 1, wherein the compound is a compound of Formula 1A: 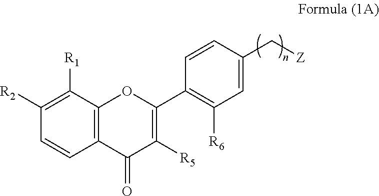
wherein:
Z is —[P(R¹¹)₃]X, wherein X is a counter anion;
R¹, R², and R⁵, independently, are selected from —OH, —O—C₁₋₄ alkyl, —OC(O)R₁₃, —OC(O)NHR¹³, —OC(O)N(R¹³)₂; or
R¹ and R² together form —O—(C₁₋₃ alkylene)-O—; and R⁵ is selected from —OH, —O—C₁₋₄ alkyl, —OC(O)R₁₃, —OC(O)NHR¹³, and —OC(O)N(R¹³)₂;
R⁶ is selected from H; halo; —CN; —NO₂; —R^{β}; —OH, —OR^{β}; —SH; —SR^{β}; —SOR^{β}; —SO₂H; —SO₂R^{β}; —SO₂NH₂; —SO₂NHR^{β}; —SO₂N(R^{β})₂; —NH₂; —NHR^{β}; —N(R^{β})₂; —CHO; —COR^{β}; —COOH; —COOR^{β}; —OCOR^{β}; and benzyl optionally substituted with 1-3—R^{β};
each —R^{β} is independently selected from a C₁-C₆ alkyl, C₂-C₆ alkenyl, C₂-C₆ alkynyl or C₃-C₁₄ cyclic group, and wherein any —R^{β} may optionally be substituted with one or more C₁-C₄ alkyl, C₁-C₄ haloalkyl, C₃-C₇ cycloalkyl, —O(C₁-C₄ alkyl), —O(C₁-C₄ haloalkyl), —O(C₃-C₇ cycloalkyl), halo, —OH, —NH₂, —CN, —NO₂, —C≡CH, —CHO, —CON(CH₃)₂ or oxo (═O) groups;
each —R¹¹ is independently selected from H, C₁-C₆ alkyl, C₂-C₆ alkenyl, C₃-C₁₄ aryl group, or C₃-C₁₄ aliphatic cyclic group, and wherein any —R¹¹ may optionally be substituted with one or more C₁-C₄ alkyl, C₁-C₄ haloalkyl, C₃-C₇ cycloalkyl, —O(C₁-C₄ alkyl), —O(C₁-C₄ haloalkyl), —O(C₃-C₇ cycloalkyl), halo, —OH, —NH₂, —CN, —C≡CH or oxo (═O) groups
each —R¹³ is independently selected from a H, C₁-C₆ alkyl, C₂-C₆ alkenyl, C₂-C₆ alkynyl, C₃₋₁₄ cyclic group, halo, —NO₂, —CN, —OH, —NH₂, mercapto, formyl, carboxy, carbamoyl, C₁₋₆ alkoxy, C₁₋₆ alkylthio, —NH(C₁₋₆ alkyl), —N(C₁₋₆ alkyl)₂, C₁₋₆ alkylsulfinyl, C₁₋₆ alkylsulfonyl, or arylsulfonyl, wherein any —R¹³ may optionally be substituted with one or more —R¹⁴;
each R¹⁴ is independently selected from a C₁-C₆ alkyl, C₂-C₆ alkenyl, C₂-C₆ alkynyl, C₃₋₁₄ cyclic group, halo, —NO₂, —CN, —OH, —NH₂, mercapto, formyl, carboxy, carbamoyl, C₁₋₆ alkoxy, C₁₋₆ alkylthio, —NH(C₁₋₆ alkyl), —N(C₁₋₆ alkyl)₂, C₁₋₆ alkylsulfinyl, C₁₋₆ alkylsulfonyl, or arylsulfonyl, wherein any —R₁₄ may optionally be substituted with one or more —R₁₅;
each —R¹⁵ is independently selected from halogen, nitro, cyano, hydroxy, trifluoromethoxy, trifluoromethyl, amino, formyl, carboxy, carbamoyl, mercapto, sulfamoyl, methyl, ethyl, methoxy, ethoxy, acetyl, acetoxy, methylamino, ethylamino, dimethylamino, diethylamino, N-methyl-N-ethylamino, acetylamino, N-methylcarbamoyl N-ethylcarbamoyl N,N-dimethylcarbamoyl, N,N-diethylcarbamoyl, N-methyl-N-ethylcarbamoyl, methylthio, ethylthio, methylsulfinyl, ethylsulfinyl, mesyl ethylsulfonyl, methoxycarbonyl, ethoxycarbonyl, N-methylsulfamoyl N-ethylsulfamoyl N,N-dimethylsulfamoyl N,N-diethylsulfamoyl, N-methyl-N-ethylsulfamoyl, carbocyclyl, aryl, or heterocyclyl; and
n=1-10.

3. A compound as claimed in claim 1 or claim 2, wherein Z is —[P(R¹¹)₃]X, wherein each —R¹¹ is independently a C₃-C₁₄ aryl group; and wherein any —R¹¹ may optionally be substituted with one or more C₁-C₄ alkyl, halo, —OH, —NH₂, —CN, —C≡CH or oxo (═O) groups.

4. A compound as claimed in any one or more of the preceding claims, wherein each R¹¹ is phenyl.

5. A compound as claimed in any one or more of the preceding claims, wherein n is 3-6, or n is 3 or 4.

6. A compound as claimed in any one or more of the preceding claims, wherein R³, R⁴, R⁶, R⁷, R⁸, and R⁹, independently, are selected from H; halo; —CN; —NO₂; —R^{β}; —SH; —SR^{β}; —SOR^{β}; —SO₂H; —SO₂R^{β}; —SO₂NH₂; —SO₂NHR^{β}; —SO₂N(R^{β})₂; —NH₂; —NHR^{β}; —N(R^{β})₂; —CHO; —COR^{β}; —COOH; and —COOR^{β}; and benzyl optionally substituted with 1-3—R^{β}.

7. A compound as claimed in any one or more of the preceding claims, wherein R³, R⁴, R⁶, R⁷, R⁸, and R⁹, independently, are selected from H; halo; —CN; —NO₂; —R^{β}; —NH₂; —NHR^{β}; —N(R^{β})₂; —CHO; —COR^{β}; —COOH; —COOR^{β}; and —OCOR^{β}.

8. A compound as claimed in any one or more of the preceding claims,
wherein R³, R⁴, R⁶, R⁷, R⁸, and R⁹, independently, are selected from H; halo; —CN; —NO₂; —R^{β}; —NH₂; —NHR^{β}; —N(R^{β})₂; and —CHO.

9. A compound as claimed in any one or more of claims 1 to 6, wherein R³, R⁴, R⁶, R⁷, R⁸, and R⁹, independently, are selected from H; halo; —CN; —NO₂; —SH; —SO₂H; and —NH₂.

10. A compound as claimed in any one or more of the preceding claims, wherein R¹, R², and R⁵, independently, are selected from —OH, —O—C₁₋₄ alkyl, and —OC(O)R₁₃.

11. A compound as claimed in any of the preceding claims, wherein R¹, R², and R⁵, independently, are selected from —OH, and —O—C₁₋₄ alkyl.

12. A compound as claimed in claim 9, wherein R¹, R², and R⁵, independently, are selected from —OH, —O—C₁₋₄ alkyl, —OC(O)R₁₃, —OC(O)NHR¹³, —OC(O)N(R¹³)₂; and R³, R⁴, R⁶, R⁷, R⁸, and R⁹, independently, are selected from H; halo; —CN; —NO₂; —SH; —SO₂H; and —NH₂.

13. A compound as claimed in claim 1 selected from the following:

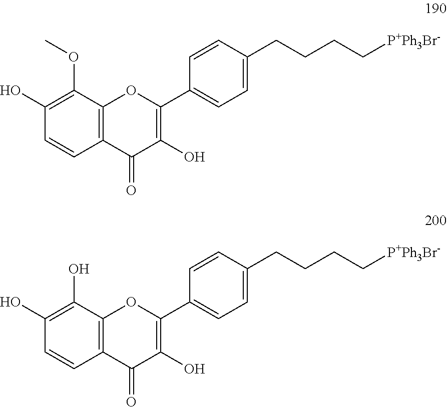

14. A pharmaceutically acceptable multi-salt, solvate or prodrug of a compound as defined in any one of claims 1 to 13.

15. A pharmaceutical composition comprising a compound as defined in any one of claims 1 to 13, or a pharmaceutically acceptable multi-salt, solvate or prodrug as defined in claim 14, and a pharmaceutically acceptable excipient.

16. A compound as defined in any one of claims 1 to 13, or a pharmaceutically acceptable multi-salt, solvate or prodrug as defined in claim 14, or a pharmaceutical composition as defined in claim 10, for use in medicine.

17. A compound as defined in any one of claims 1 to 13, or a pharmaceutically acceptable multi-salt, solvate or prodrug as defined in claim 14, or a pharmaceutical composition as defined in claim 15, for use treating or preventing cancer.

18. A method of treatment or prevention of a disease, disorder or condition, the method comprising the step of administering an effective amount of a compound as defined in any one of claims 1 to 13, or a pharmaceutically acceptable multi-salt, solvate or prodrug as defined in claim 14 or a pharmaceutical composition as defined in claim 15, to thereby treat or prevent the disease, disorder or condition.

19. A method of treatment as claimed in claim 18, wherein the disease, disorder or condition is cancer.

20. A compound, a pharmaceutically acceptable multi-salt, solvate or prodrug, or a pharmaceutical composition, for use according to claim 17, or a method of treatment according to claim 19, wherein the cancer is brain cancer, breast cancer, colon cancer, leukaemia, lung cancer, lymphoma, ovarian cancer, pancreatic cancer, prostate cancer, renal cancer and skin cancer (melanoma).
